(19) United States
(12) Patent Application Publication
(10) Pub. No.: US 2010/0152523 A1
(43) Pub. Date: Jun. 17, 2010

(54) Method and Apparatus for Minimally Invasive Direct Mechanical Ventricular Actuation

(75) Inventors: Stuart G. MacDonald, PULTNEYVILLE, NY (US); Ruheri Anthony Perez-Tamayo, LaGrange, IL (US); George w. Anstadt, Pittsford, NY (US); Mark P. Anstadt, Dayton, OH (US)

Correspondence Address: BUCHANAN, INGERSOLL & ROONEY PC, POST OFFICE BOX 1404, ALEXANDRIA, VA, 22313-1404, US

(73) Assignee: MYOCARDIOCARE, INC., PITTSFORD, NY (US)

(21) Appl. No.: 12/085,547

(22) PCT Filed: Nov. 28, 2006
(86) PCT No.: PCT/US2006/045492

§ 371 Date: May 27, 2008

Related U.S. Application Data
(60) Provisional application No. 60/739,945, filed on Nov. 28, 2005.

(51) Int. Cl. A61N 1/362

(52) U.S. Cl. 600/16

(57) ABSTRACT

Disclosed is a device for assisting the function of a heart that is collapsible to facilitate minimally invasive procedures. The cup-shaped device may be inserted into the chest cavity and deployed on the heart via a specially configured tube. The device comprises a cup-shaped shell expanded by a support cage disposed within the shell, and an elastic cup-shaped liner, together forming an inflatable cavity between the outer surface of the liner and the inner surface of the shell. Alternate application of positive and negative pressures to the cavity provides controlled, active, systolic and diastolic support to the heart.

### TECHNICAL FIELD

Mechanical devices are described which assist a heart in providing proper systolic and diastolic circulatory function, and which are capable of being placed on the heart in a minimally invasive manner.

### BACKGROUND ART

Traditional medical and surgical treatment of patients with failing pump function of the heart is mostly limited to blood-contacting devices that are technically difficult to install and result in complications related to such blood contact as well as technical aspects of device installation. Inadequate cardiac output remains a cause of millions of deaths annually in the United States. Mechanical devices are proving to be a practical therapy for some forms of sub-acute and chronic low cardiac output. However, all currently available devices require too much time to implant to be of value in acute resuscitation situations, resulting in loss of life before adequate circulatory support can be provided.

Mechanical cardiac assistance devices are also known which generally operate by providing blood pumping support to the circulation to assist the failing heart. A number of mechanical techniques for assisting heart function by compressing its outer epicardial surface have been described and studied. These methods have focused on improving cardiac performance by assisting the systolic (positive pumping) function of the heart. Such techniques have been described as “direct cardiac compression” (DCC). DCC methods have been investigated only in the laboratory setting, and there are no uses of such devices in human subjects known to the applicants. Examples of DCC techniques include, but are not limited to, cardiomyoplasty (the technique of wrapping skeletal muscle around the heart and artificially stimulating it), the “Cardio Support System” (Cardio Technologies, Inc., Pinebrook, N.J.) and the “Heart Booster” (Abiomed, Inc., Danvers, Mass.). Cumulative results from laboratory investigations using these devices have resulted in similar findings. Specifically, DCC has been shown to enhance left ventricular (LV) pump function without any apparent change in native LV oxygen consumption requirements; thereby, DCC has been shown to improve LV pump function without increasing myocardial oxygen consumption and/or requiring extra work from the heart.

DCC devices have been shown to only benefit hearts with substantial degrees of LV failure. Specifically, DCC techniques only substantially improve the systolic function of hearts in moderate to severe heart failure. In addition, the benefits of DCC techniques are greater when applied to the relatively dilated or enlarged LV. Therefore, the relative degree of assistance provided by DCC improves as heart failure worsens and the heart enlarges or dilates from such failure. DCC techniques clearly have a negative effect on diastolic function (both RV and LV diastolic function). This is exhibited by reductions in diastolic volume that, in part, explains DCC's inability to effectively augment the heart without at least moderate degrees of failure. This also explains DCC's efficacy being limited to sufficient degrees of LV size and/or dilatation, with significant dependence on preload, and/or ventricular filling pressures. Thus, DCC requires an “adequate” degree of heart disease and/or heart failure to benefit the heart's function. In addition, DCC devices have negative effects on the dynamics of diastolic relaxation and, in effect, reduce the rate of diastolic pressure decay (negative dP/dt max), increasing the time required for ventricular relaxation. This better explains why DCC techniques require substantial degrees of LV and RV loading (i.e. increased left and right atrial pressure or “preload”) to be effective, as such increases serve to augment ventricular filling. This latter point is particularly true with smaller heart size and/or less ventricular distension.

The critical drawbacks to DCC methods are multi-factorial and are, in part, summarized in the following discussion. First, and foremost, these techniques do not provide any means to augment diastolic function of the heart necessary to overcome their inherent drawback of “effectively” increasing ventricular stiffness. This is illustrated by the leftward shifts in the end-diastolic pressure-volume relationship (EDPVR) during DCC application. This effect on the EDPVR is seen with DCC devices in either the assist or non-assist mode. Clearly, RV diastolic function is impaired to a far greater degree by DCC due to the nature both the RV wall and intra-cavity pressures. Furthermore, studies of DCC devices have typically overlooked the relevant and dependent impact these techniques have on right ventricular dynamics, septal motion and overall cardiac function. Because the right ventricle is responsible for providing the “priming” blood flow to the left ventricle, compromising right ventricular function has a necessary secondary and negative impact on left ventricular pumping function when these load-dependent devices are utilized. Furthermore, the ventricular septum lies between the right and left ventricle and is directly affected by the relevant forces placed on both the RV and LV. Another disadvantage of known DCC devices can be an inability to continuously monitor ventricular wall motion and chamber dynamics that are intuitively critical to optimizing the assist provided by such mechanical actions on the right and left ventricular chambers which behave in a complex, inter-related fashion. Additionally, studies regarding known DCC methods tend not to adequately examine the effects of these devices on myocardial integrity.

The Direct Mechanical Ventricular Assist device (hereinafter abbreviated as DMVA) is an example of one type of mechanical cardiac assistance device. In general, a DMVA system comprises two primary elements: (a) a Cup device having dynamic characteristics and material construction that keep the device's actuating liner membrane or diaphragm closely conformed to the exterior surface (or epicardium) of the heart throughout systolic and diastolic actuation, and (b) a Drive system and control system combination that cyclically applies hydraulic pressure to a compression and expansion liner membrane or membranes located on the interior surfaces of the Cup in a manner that augments the normal pressure and volume variations of the heart during systolic and diastolic actuation. The cyclic action of the cup device (hereinafter referred to simply as “the Cup”) cyclically pushes and pulls on the left and right ventricles of the heart.

By providing this cyclic motion at the appropriate frequency and amplitude, the weakened, failing, fibrillating, or asystolic heart is driven to pump blood in a manner which approximates blood flow generated by a normally functioning heart. Pushing inwardly on the exterior walls of the heart compresses the left and right ventricles into systolic configuration(s), thereby improving pump function. As a result, blood is expelled from the ventricles into the circulation. Immediately following each systolic actuation, the second phase of the cycle applies negative pressure to the liner membrane to return the ventricular chambers to a diastolic configuration by pulling on the outer walls of the heart. This is termed diastolic actuation and allows the ventricular chambers to refill with blood for the next compression.

Commonly, in installing the Cup, the heart is exposed by a chest incision, such as a sternotomy or a thoracotomy. The Cup is then positioned over the apex of the heart in a position such that the apex of the heart is partially inserted therein. A vacuum is applied to the apex of the Cup, thereby pulling the heart and the Cup together, such that the apices of the Cup and the heart, and the inner wall of the Cup and the epicardial surface of the heart become substantially attached. Connections are then completed for any additional sensing or operational devices (typically integrated into a single interface cable) if the particular DMVA includes such devices. This procedure can be accomplished in minutes, and it is easy to teach to individuals with minimal surgical expertise.

However, the sternotomy and the thoracotomy are considered to be highly invasive and traumatic to the patient. A more preferred approach is to access the heart from below the chest cavity, and deploy the DMVA cup over the heart at the apex of the heart. Such an approach is more suitable to minimally invasive surgical procedures, and to the use of minimally invasive surgical tools developed specifically for such procedures.

Effective DMVA requires that the Cup and Drive system satisfy multiple and complex performance requirements.

Known DMVA devices are not capable of being installed on the heart via a minimally invasive procedure, and/or are incapable of providing the desired operational features, including integrated heart parameter sensing, therapeutic agent delivery, and/or remodeling capability via device-control algorithms. There is a need for a DMVA with such features that can be installed by a minimally invasive surgical procedure. There is also a need for tools designed to deploy such a device on a heart through a minimally invasive surgical procedure. There is also a need for to accomplish the deployment of the device very quickly, in order to avoid ischemia, brain death, and other organ damage, particularly where cardiac arrest has occurred.

### SUMMARY

A DMVA device is provided for assisting in a body the function of a heart in a body. The device is configured to be installed in the patient's body using a surgical procedure that does a minimal amount of damage to nearby tissues. This is made possible by making the device highly collapsible to a compact shape, and self-expanding from the compact shape. When the device is collapsed, it can be placed in a tool that holds it in a collapsed shape. The tool may be an elongated tube that holds the collapsed device inside. To install the device on the heart of a patient, a small incision is made in the chest cavity of the patient, the tube holding the collapsed device is inserted into the incision and brought to the apex of the heart. The device is pushed forward out of the tube, and springs open, expanding and enveloping the heart. The device is then connected to a control system and begins to provide assistance to the heart.

In one embodiment, the device comprises a cup-shaped shell having a longitudinal axis, and comprising a wall formed from a polymer-fiber composite and having an outer surface and an inner surface, the wall extending from a hole at an apex of the shell to a rim optionally including an annular chamber; a cup-shaped support cage disposed within and joined to the cup-shaped shell, the support cage comprising a base comprising a port and a plurality of flexible radial struts comprising proximal ends extending from the base contiguously along the inner surface of the shell to distal ends that are proximate to the rim of the shell; a liner comprising an outer surface, an inner surface, an upper region joined to the rim of the cup-shaped shell, a tapered seal extending inwardly from the upper region toward the longitudinal axis of the cup-shaped shell, an elastic central region, and a lower region joined to the cup-shaped shell proximate to the base of the support cage, thereby forming an inflatable cavity between the outer surface of the liner and the inner surface of the shell; a first fitting connected to the apex of the cup-shaped shell and connectable to a first lumen; and; and a second fitting in communication with the inflatable cavity between the outer surface of the liner and the inner surface of the shell and connectable to a second lumen.

The fitting for connecting to the first lumen may comprise a tubular body, a flared proximal end engaged with the inner surface of the liner and passing through a hole in the inner surface of the liner at the lower region of the liner, an engagement lip for engagement with the tapered port of the cup-shaped support cage, a distal end, and a passageway extending from the distal end of the fitting to the flared proximal end of the fitting. In one embodiment, the fitting may be formed integrally with the liner. In another embodiment, the fitting for connecting to the first lumen and the fitting for connecting to the second lumen may be integrated into a single unitary fitting, comprising a first passageway to render the first lumen in fluid communication with the inner volume of the device and a second passageway to render the second lumen in fluid communication with the inflatable cavity between the outer surface of the liner and the inner surface of the shell. This unitary fitting may be comprised of a tubular body that may be cylindrical or oblong in shape, a flared proximal end engaged with the inner surface of the liner and passing through a hole in the inner surface of the liner at the lower region of the liner, an engagement lip for engagement with the port of the cup-shaped support cage, a distal end, a first passageway extending from the distal end of the fitting to the flared proximal end of the fitting, and at least a second passageway extending from the distal end of the fitting to a third passageway in the lower region of the liner, the third passageway extending from the second passageway in the fitting to the inflatable cavity between the outer surface of the liner and the inner surface of the shell. The first passageway extending from the distal end of the fitting to the flared proximal end of the fitting may be aligned with the longitudinal axis of the cup shaped shell. The unitary fitting may further comprise a plurality of radially disposed passageways spaced around the first passageway and in communication with a plurality of liner passageways extending through the lower region of the liner to the inflatable cavity between the outer surface of the liner and the inner surface of the shell. The unitary fitting may be formed integrally with the liner.

The cup-shaped support cage may be joined to the cup-shaped shell by adhesive or by embedding the cup-shaped support cage within a coating disposed on the inner surface of the cup-shaped shell. The number of the radial struts of the cup-shaped support cage may be between 8 and 32, and in one embodiment, the number of the radial struts of the cup-shaped support cage may be 16. The distal ends of the radial struts may extend to the annular chamber of the rim of the cup-shaped shell. At least one of the radial struts of the cup-shaped support cage comprises an engagement feature at the distal end thereof, such as a T-shape at the distal end thereof.

The device is elastically deformable so that it can be collapsed along its longitudinal axis and introduced to the interior of a chest cavity and onto the heart through a small incision. In one embodiment, the device is introduced via a deployment tool providing access to the interior of the chest cavity through the incision. The collapsed device is passed through the deployment tool, and, when proximate the heart within the chest cavity, the device is restored to its original expanded shape to facilitate deployment onto the heart. Thus, as used herein, the terms “elastically deformable” and/or “elastic deformation” refer to the capacity to collapse a structure and restore it to its original shape without loss of structural integrity or strength.

In one embodiment, the device is deformable such that it is collapsed and folded around its longitudinal axis to a generally rod-shaped structure having a diameter of about 4 cm or less. In some embodiments, the collapsed device will have a diameter of about 2.5 cm or less; and in still another embodiment, it will have a collapsed diameter of about 2 cm. One of skill in the art will appreciate that the selection of materials will play a role in the degree of deformability, and the ultimate diameter of the collapsed device. Likewise, the size, shape and contours of the collapsed device will inform the selection and configuration of the deployment tool. Generally, the deployment tool will assume a tubular, but not necessarily strictly circular, structure or internal configuration. The diameter of the deployment tool will be complementary to the configuration and diameter of the collapsed device to ensure that the device is readily passed through the tool.

To facilitate elastic deformation of the device, the struts of the support cage can be fabricated from deformable high strength metal alloys. Nonlimiting examples include titanium and/or tantalum, and their various alloys. Titanium alloys useful in such embodiments include the high strength shape memory alloys, including those comprising nickel and titanium (e.g., various members of the class of alloys commercially available as nitinol). Both titanium and tantalum alloys have the dual advantage of high strength and low magnetic susceptibility, which creates minimal image artifact in Magnetic Resonance Imaging (MRI) and Magnetic Resonance Angiography (MRA). Stainless steel alloys can also be used; however, many such alloys have magnetic susceptibility due to the presence of iron, chromium, etc., which contributes to image artifact in MRI and MRA. Those alloys might still be used where MRI and/or MRA are not contemplated; or resort may be had to commercially available “non-magnetic” stainless steels that produce little or no MRI/MRA image artifact. Other flexible metal alloys such as blue tempered and polished steel (also known as clock spring steel) having a carbon content of between about 0.90 to 1.04 percent and a Rockwell hardness of about C48 to C51.

Still other suitable materials for the formation of the struts include carbon fiber composites and/or other composites, such as aramid fiber (e.g., KEVLAR® brand fibers commercially available from DuPont; and TWARON® brand fibers from Teijin Co.), and glass fiber. The matrix materials of such composites can be a polymer such as an epoxy matrix, a ceramic matrix, or a metal. Ultimately, one of skill in the art will appreciate that suitable materials include those that are spring-like (stiff and flexible), having a high modulus of elasticity (stiffness) and a suitable yield point (degree of stretch or bending at failure).

The support cage must also be fabricated to resist significant collapsing during diastolic assistance to the heart. When vacuum is applied to the elastic liner of the device to provide diastolic assistance, the support cage prevents inward flexing of the cup-shaped shell. Since the internal volume of the device is thus maintained during vacuum application, the elastic liner is pulled outwardly toward the wall of the cup shaped shell, thereby pulling outwardly on the walls of the heart ventricles and providing diastolic assistance.

The polymer-fiber composite of the cup-shaped shell must also be collapsible, but selected and constructed such that it provides little or substantially no resistance to collapsing of the device for placement in a deployment tube. In contrast, when the support cage is fully open and the device is deployed on a heart, the cup-shaped shell prevents any significant expansion of the internal volume of the device. The polymer fiber composite that forms the shell must be flexible such that the shell can be folded when the device is collapsed, but also inelastic when placed under tension, so that the internal volume is constrained when the device is assisting a heart. In particular, during systolic assistance to a heart, when fluid pressure is applied to the elastic liner of the device, the polymer fiber composite is in tension and prevents an increase in the internal volume of the device. Since the cup shell internal volume cannot increase, the elastic liner is forced to deform and stretch inwardly, thereby displacing the walls of the heart ventricles and providing systolic assistance.

An example of a polymer-fiber composite with sufficient flexibility and tensile strength is a polyester fiber such as DACRON™ fiber, and/or a polyurethane polymer. The fiber of the polymer fiber composite may be wound circumferentially around the shell to form a fiber matrix of substantially uniform fiber density. Alternatively, the fiber of the polymer fiber composite may be chopped fiber forming a fiber matrix of substantially uniform fiber density, or the fiber of the polymer fiber composite may be formed in a woven mesh fabric.

The liner of the device may be a silastic elastomer or any similarly biocompatible polymer with analogous elastomeric properties. The elastic central region of the liner is deformable by fluid pressure such that the volume of the inflatable cavity may be varied between about zero at end diastole (largest heart size) and about 175 cubic centimeters at end systole (fully compressed heart). This is a nominal value and may range from less than 75 cubic centimeters for a small size cup delivering partial pumping assist to the heart, to more than 250 cubic centimeters for a large size cup delivering full assist to the heart at a relatively low pulse rate. In general, the volume of the inflatable cavity may be varied between the amounts of cardiac output that may be desired in a patient in any given physiologic state.

In one embodiment, the elastic central region of the liner is deformable by fluid pressure within the inflatable cavity of between about −40 millimeters of mercury and about 140 millimeters of mercury within the inflatable cavity without rupture. Additionally, when the inflatable cavity is subjected to a variation of internal gauge pressure of between about −40 millimeters of mercury and about 140 millimeters of mercury, the volume enclosed within the wall of the cup-shaped shell varies by less than about 5 to 10 percent of the volume enclosed at zero gauge pressure. A cup of this design having few struts in the support cage will result in relatively large movement of the compliant wall between the struts during the systolic-to-diastolic pressure change. For a support cage having an infinite number of struts, this movement of the compliant wall will approach zero. A practical device design will find a compromise that provides small overall cross-section when collapsed for delivery, sufficient bending rigidity of struts, and relatively small movement of the compliant wall during systolic-to-diastolic pressure change. The range of this movement, representing additional pumping requirements for the Drive Unit to deliver proper DMVA support, will be between 4% and 10% and will depend on the design factors outlined above. The size of the cup shaped shell may be varied in different embodiments to accommodate the largest or smallest human hearts. The cup shaped shell may have a maximum diameter of between about 80 and about 140 millimeters and the distance along the longitudinal axis from the apex to the rim may be made approximately equal to the diameter of the cup shaped shell.

The liner may also be provided such that the inflatable cavity is formed entirely within the liner, instead of between the outer surface of the liner and the inner surface of the shell. In this embodiment, the liner is comprised of an upper region joined to the rim of the cup-shaped shell, a tapered seal extending inwardly from the upper region toward the central axis of the cup-shaped shell, an elastic central region including an inflatable cavity formed in the elastic central region, and a lower region joined to the cup-shaped shell proximate to the base of the support cage.

There is also provided a tool for implanting in a body a DMVA device using minimally invasive procedures, the tool comprising an elongated tube, a piston disposed in the elongated tube and operably connected at a lower surface to a plunger and a knob. The piston is bidirectionally movable within the bore of the tube, and the tube is adapted to receive the collapsed DMVA device within the bore thereof, for subsequent deployment onto a heart. The piston may be provided with a cavity in the upper surface thereof adapted for receiving a fitting of the DMVA device during loading of the device in the bore thereof; and subsequent deployment therefrom onto a heart. The piston, plunger, and knob of the device may be provided with a bore therethrough such that a lumen may be fed therethrough and connected to the DMVA device for the purpose of providing a vacuum within the device to assist in deployment onto a heart. There is further provided a loading tool assembly for loading a DMVA device into the tool for implanting the DMVA device in a body. The loading tool assembly comprises a funnel including a tapered conical section. The tapered conical section may be joined to a short neck section. The short neck section may be dimensioned so as to be engageable with the bore of the tool for implanting the DMVA device in a mild interference fit. The loading tool may further include a guide wire for pulling the DMVA device into the bore of the implanting tool. The funnel may be fluted, and the number of flutes may be equal to the number of struts of the support cage of the DMVA device, if the DMVA device is provided with such struts. There is further provided a method for deploying a DMVA device onto a heart in a minimally invasive procedure comprising the steps of loading the device into the bore of the deployment tool, accessing the heart through an incision in the thorax, inserting the tool containing the DMVA device through the incision, and deploying the device from the tool onto the heart.

There is also provided an access tool for gaining access to the apex of the heart through the pericardium, and subsequently deploying a DMVA device onto a heart in a minimally invasive surgical procedure. The access tool is comprised of a tubular housing, a retainer cap, a suction tube assembly, a cutting sleeve, and a retainer sleeve. The retainer sleeve is joined at its upper and lower ends to the tubular housing by known materials joining structures such as spot welds. The cutting sleeve is disposed in an annular gap formed between the retainer sleeve, and the tubular housing, and includes cutouts located at the spot welds so as to render the cutting sleeve movable axially and rotationally around the tubular housing. The cutting sleeve further includes cutting blades at the distal end thereof. When the cutting sleeve is withdrawn into the annular gap, the cutting blades are splayed open, providing a circular opening for the advancement of the suction tube assembly. The suction tube assembly includes a tube for applying vacuum to a suction cup, which is attachable to the pericardium during the surgical access procedure. The suction cup is used to pull the pericardium away from the heart, and the cutting blade assembly is subsequently advanced such that the circularly arranged cutting blades may be used to cut through the pericardium. The cutting blades may then be further spread, stretching the opening through the pericardium wider, beyond the diameter of the tubular housing. The retainer cap is removable, such that the suction tube assembly may be removed, and replaced with a DMVA device deployment assembly comprising another retainer cap, a deployment sleeve, a piston, and a plunger rod. A collapsed DMVA device may be contained in the deployment sleeve. The reassembled tool thus resembles the previously described DMVA deployment tool described previously, and is thus suitable for delivery of a DMVA device after being used to access the heart through the pericardium.

The DMVA device described above is advantageous because it precisely drives the mechanical actuation of the ventricular chambers of the heart without damaging the tissue thereof, or the circulating blood; while being installed by a simple minimally invasive procedure that can be quickly performed. Embodiments of the DMVA device may monitor and provide functional performance and/or image data of the heart; and/or electrophysiological monitoring and control of the heart, including pacing and cardioversion-defibrillation electrical signals to help regulate and/or synchronize device operation with the native electrical rhythm and/or contractions thereof. As a result, a greater variety of patients with cardiac disease can be provided with critical life-supporting care in a minimally invasive manner, under a greater variety of circumstances, including but not limited to, resuscitation, bridging to other therapies, and extended or even permanent support.

Also provided is a method of deploying a minimally invasive DMVA. In one method, the shell is collapsed from an open cup-shape to a compact configuration that is collapsed along the shell's longitudinal axis. In one embodiment, the shell is introduced via a deployment tool, which may be a flexible or rigid hollow structure compatible with the longitudinally collapsed shell, e.g., tubular. A modest sized incision may then be made proximate the heart. In one embodiment, the incision is made in the chest, and may be positioned to facilitate insertion of the deployment tool between the ribs or below the rib cage. The deployment tool is inserted into the incision, whereupon the collapsed shell is displaced from the deployment tool. Upon displacement, the collapsed shell resumes its open, or cup-shaped, configuration. In one embodiment, the interior of the cup-shaped shell complements the shape of the heart requiring assistance. The open cup-shaped shell is then positioned over the heart. The DMVA is then positioned to assist the function of the heart, by structurally supporting systolic and/or diastolic action, and/or by regulating the timing thereof.

The DMVA device can support the heart through a period of acute injury and allow healing that potentially results in substantially a full recovery of unsupported heart function.

### BRIEF DESCRIPTION OF THE DRAWINGS

The invention will be described by reference to the following drawings, in which like numerals refer to like elements, and in which:

FIG. 1 is a perspective view of an embodiment of a DMVA device that is capable of being deployed onto a heart by a minimally invasive surgical procedure;

FIG. 2A is a side cross-sectional view of the DMVA device of FIG. 1 comprising a liner having a single-wall central elastic region that forms an inflatable cavity in cooperation with the shell wall of the device, shown in a state of completing assistance of diastolic expansion of a heart, taken along the lines 2A/B-2A/B of FIG. 1;

FIG. 2B is a side cross-sectional view of the DMVA device of FIG. 1 and

FIG. 2A shown in a state of completing assistance of systolic compression of a heart, taken along the line 2A/B-2A/B of FIG. 1;

FIG. 3 is an exploded perspective view of the DMVA device of FIG. 1;

FIG. 4A is a perspective view of the support cage of the DMVA device of FIGS. 1-3;

FIG. 4B is a side elevation view of the support cage of FIG. 4A;

FIG. 4C is a top view of the support cage of FIGS. 4A and 4B, taken along line 4C-4C of FIG. 4B;

FIG. 4D is a top view of a support cage similar to the cage of FIGS. 4A-4C, but provided with an elliptical central hole for engagement with the unitary fluid fitting of FIGS. 7A and 7B;

FIG. 5A is a perspective view of the shell of the DMVA device of FIGS. 1-3;

FIG. 5B is a side cross-sectional view of the shell of the DMVA device of FIGS. 1-3;

FIG. 6 is a detailed cross-sectional view of an embodiment of a single port fitting for connecting to a first lumen at the apex of the DMVA shell to render the first lumen in fluid communication with the inner volume of the shell;

FIG. 7A is a detailed side cross-sectional view of an embodiment of a unitary dual port fitting for connecting to a first lumen at the apex of the DMVA shell to render the first lumen in fluid communication with the inner volume of the shell and for connecting to a second lumen to render the second lumen in fluid communication with an inflatable cavity in an elastic central region of the liner;

FIG. 7B is an axial view of an embodiment of the unitary fitting of FIG. 7A, taken along line 7B-7B of FIG. 7A;

FIG. 8A is a cross-sectional view of the DMVA device of FIGS. 2A and 2B, shown assisting a heart at the completion of diastole;

FIG. 8B is a cross-sectional view of the DMVA device of FIGS. 2A and 2B, shown assisting a heart at the completion of systole;

FIG. 9 is a cross-sectional view of an embodiment of a DMVA device similar to the DMVA device of FIGS. 1-3 but fitted with the dual port fitting of FIGS. 7A and 7B, shown assisting a heart at the completion of systole;

FIG. 10A is a longitudinal cross-sectional view of the DMVA device of FIGS. 1-3 shown collapsed and inserted into an endoscopic tool for deployment onto a heart;

FIG. 10B is an axial cross-sectional view of the DMVA device and tool of FIG. 10A, taken along the line 10B-10B of FIG. 10A;

FIG. 11 is a cross-sectional view of an embodiment of a tool provided for use in loading a DMVA device into the deployment tool of FIG. 10A;

FIG. 12 is a side cross-sectional view of another embodiment of a DMVA device that is capable of being deployed onto a heart by a minimally invasive surgical procedure, shown in a state of completing assistance of systolic compression of a heart;

FIG. 13 is an exploded perspective view of the DMVA device of FIG. 12;

FIG. 14 is a longitudinal cross-sectional view of the DMVA device of FIGS. 12 and 13 shown collapsed, inverted, and temporarily attached to an endoscopic inflation tool for deployment onto a heart;

FIG. 15 is a longitudinal cross-sectional view of the DMVA device and endoscopic inflation tool of FIG. 14 shown with the collapsed DMVA device partially deployed onto a heart;

FIG. 16 is a side cross-sectional view of another embodiment of a DMVA device similar to the device of FIG. 12, shown in a state of completing assistance of systolic compression of a heart;

FIG. 17A is a side cross-sectional view of a further embodiment of a DMVA device similar to the device of FIG. 12, shown in a state of completing assistance of systolic compression of a heart;

FIG. 17B is a detailed cross-sectional view of the shell wall of the DMVA device of FIG. 17A, a portion of which is denoted by the ellipse 17B of FIG. 17A;

FIG. 18A is a side cross-sectional view of yet another embodiment of a DMVA device that is capable of being deployed onto a heart by a minimally invasive surgical procedure, shown in a state of completing assistance of diastolic expansion of a heart;

FIG. 18B is a side cross-sectional view of the DMVA device of FIG. 18A shown in a state of completing assistance of systolic compression of a heart;

FIG. 19A is a cross-sectional view of an embodiment of a tool for gaining access to the apex of the heart through the pericardium, shown immediately prior to use;

FIG. 19B is a cross-sectional view of the tool of FIG. 19A, shown pulling a portion of the pericardium from the heart, and cutting through the pericardium;

FIG. 19C is a cross-sectional view of the tool of FIG. 19A, shown as having cut through and stretched the pericardium open, in order to provide access to the heart for deployment of a minimally invasive DMVA device, or for some other beneficial purpose;

FIG. 20A is a detailed side elevation view of the portion of the access tool indicated by line 20A-20A in FIG. 19A, shown immediately prior to use;

FIG. 20B is an end view of the access tool taken along line 20B-20B of FIG. 20A;

FIG. 21A is a detailed side elevation view of the portion of the access tool indicated by line 21A-21A in FIG. 19B, shown deployed for pulling a portion of the pericardium from the heart, and cutting through the pericardium;

FIG. 21B is an end view of the access tool taken along line 21B-21B of FIG. 21A; and

FIG. 22 is a side cross-sectional view of the access tool of FIGS. 19A-21B, with the pericardial suction assembly having been removed, and replaced by a sleeve and plunger assembly containing a minimally invasive DMVA device.

### DETAILED DESCRIPTION

For a general understanding of certain embodiments of the DMVA, reference is made to the drawings, wherein like reference numerals have been used throughout to designate identical elements.

As used herein, the term Cup is meant to indicate the Direct Mechanical Ventricular Assist device as described herein, such device comprising a cup-shaped outer shell. The terms Cup, DMVA Cup, DMVA device, and DMVA apparatus may be used interchangeably in this specification and are intended to denote the overall Direct Mechanical Ventricular Assist device described herein in various embodiments, unless specifically noted otherwise. The cup-shaped outer shell comprises a container forming a curved conical void, or a substantially parabolic or hyperbolic void. In one embodiment, the void of the cup-shaped shell is complementary to the exterior ventricular portion of a human heart. The cup-shaped shell provides a support enclosure within which the ventricular region of the heart is constrained. The upper rim of the cup-shaped shell also provides a ring-shaped constraint, which may limit the size of the heart at the atrio-ventricular groove and provide a beneficial effect in the operation of the valves of the heart. The exact shape of the cup-shaped shell may be varied, depending upon the general shape of the ventricular portion of the heart to which it is being fitted. For example, a heart that is afflicted with dilated cardiomyopathy may have a more rounded shape (i.e. a lower ratio of length to diameter), and thus the shape of the cup shaped shell may be provided to better fit such a heart.

As used herein, the abbreviation LV is meant to denote the term “left ventricle”, or “left ventricular” and the term RV is meant to denote the term “right ventricle, or “right ventricular”, as appropriate for the particular context.

“Right” and “left” as used with respect to the ventricles of the heart are taken with respect to the right and left of the patient's body, and according to standard medical practice, wherein the left ventricle discharges blood through the aortic valve into the aorta, and the right ventricle discharges blood through the pulmonic valve into the pulmonary artery. However, the Figures of the instant application, which depict various embodiments of the DMVA and the heart contained therein are taken as viewed facing the patient's body. Accordingly, in such Figures, the left ventricle depicted in any such Figure is to the right, and vice-versa just as is done in convention when viewing radiographs and figures of related organs in the medical field. For the sake of clarity in such Figures, the left and right ventricles are labeled “LV” and “RV”, respectively.

As used herein, the terms “normal heart”, and “healthy heart” are used interchangeably, and are meant to depict a normal, unafflicted human heart, not in need of DMVA assistance or other medical care.

As used herein, the term cardiac function is meant to indicate a function of the heart, such as the pumping of blood in systemic and pulmonary circulation; as well as other functions such as healing and regeneration of the heart following a traumatic event such as e.g., myocardial infarction. Parameters indicative of such functions are physical parameters, including but not limited to blood pressure, blood flow rate, blood volume, and the like; and chemical and biological parameters such as concentrations of oxygen, carbon dioxide, lactate, etc.

As used herein, the term cardiac state is meant to include parameters relating to the functioning of the heart, as well as any other parameters including but not limited to dimensions, shape, appearance, position, etc.

As used herein, the term “minimally invasive” is meant to indicate with regard to a surgical procedure, the accessing of a site within the body of a patient, and optionally, the deploying an implantable device at the site in a manner that entails less disruption and damage to tissues at the site than standard surgical procedures, which entail large incisions, spreaders, clamps, and other means to access the site. More specifically, a full sternotomy is an example of an invasive surgical procedure, which involves making an incision in the middle of the chest from the top of the sternum to the bottom. This method of heart surgery is the standard approach where the entire rib cage is opened and the heart muscle is fully exposed. In contrast, examples of minimally invasive surgical procedures to access a heart in a body are a sub-xyphoid incision and approach, a subcostal incision and approach, or a mini-thoracotomy.

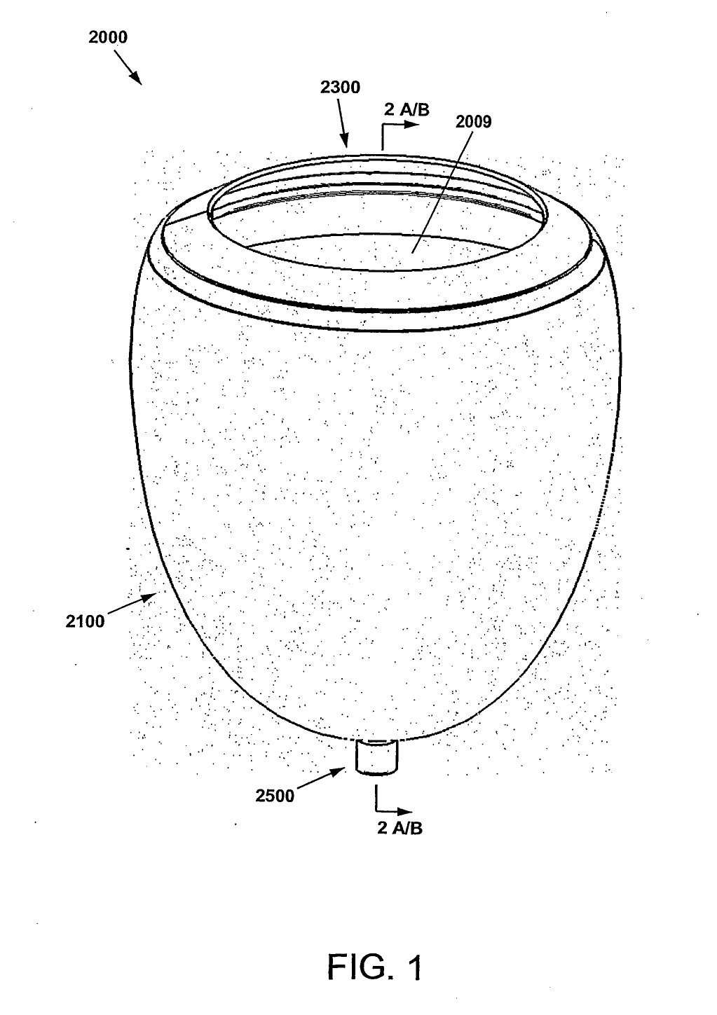

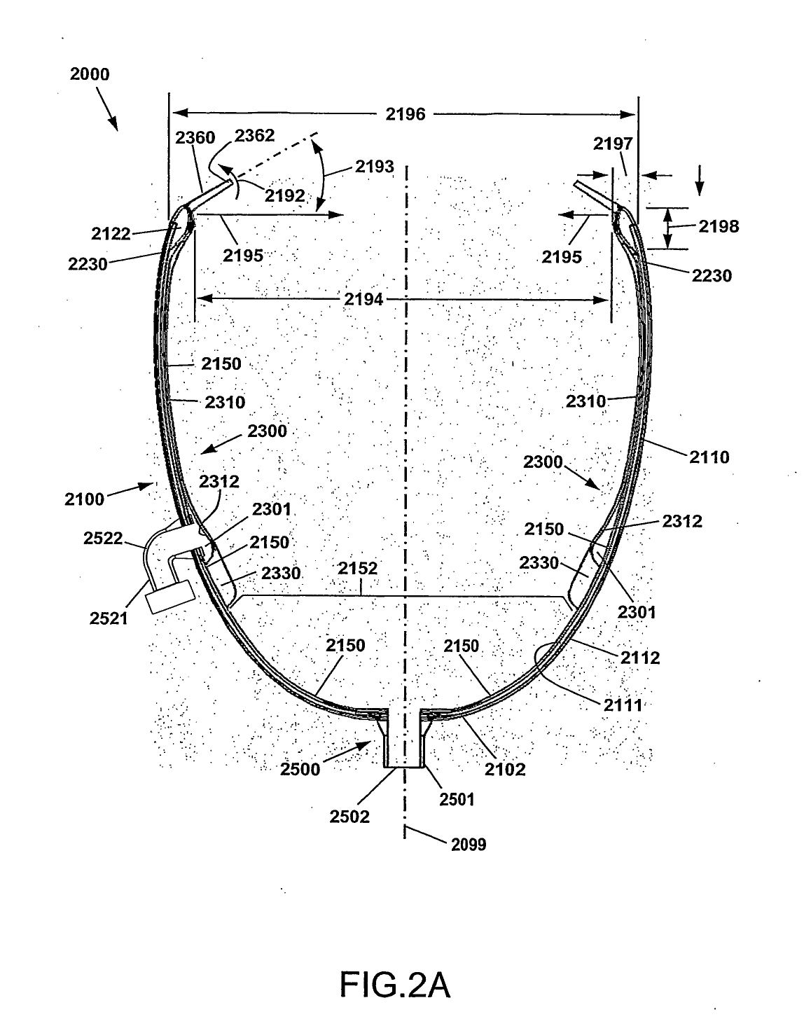

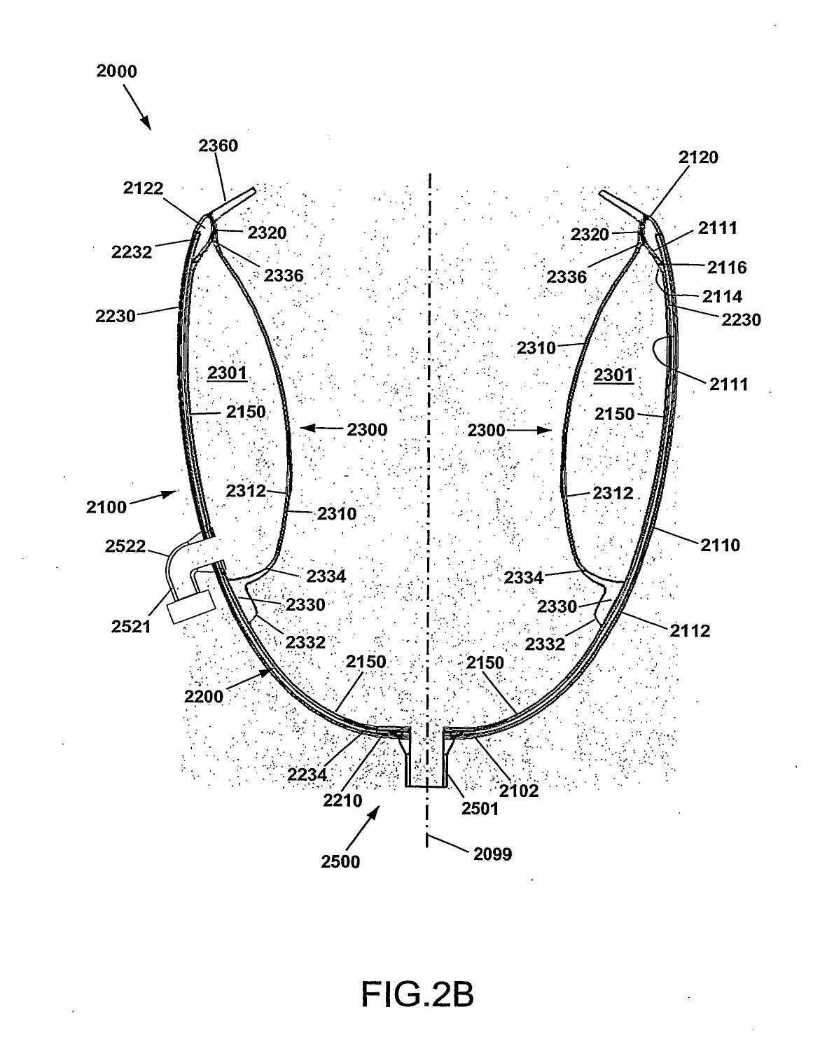

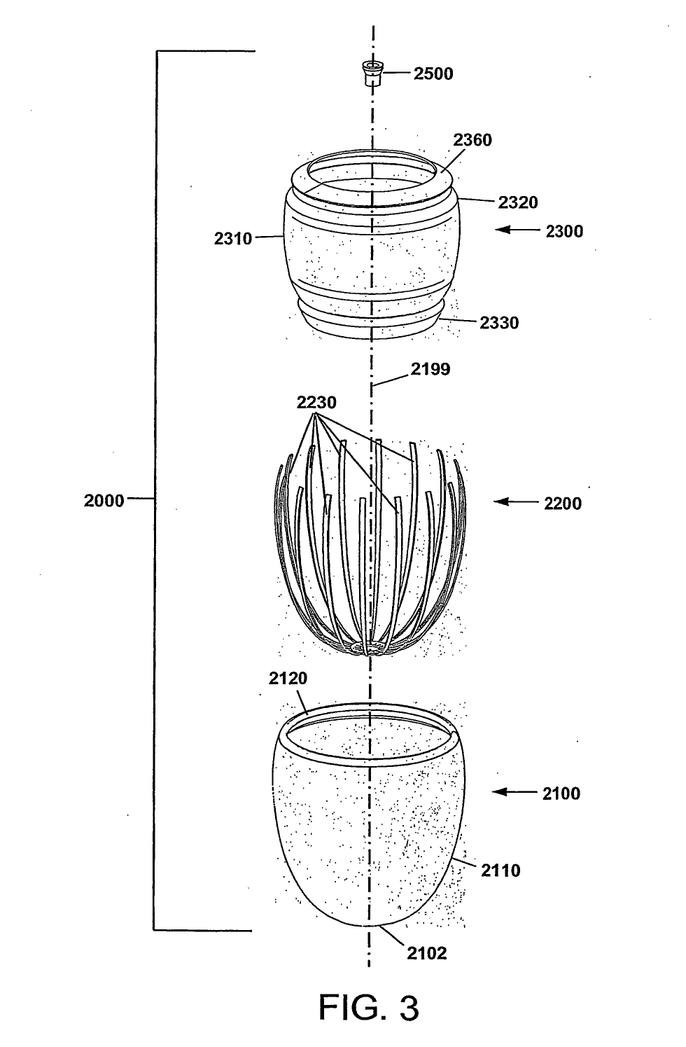

Referring to the drawings, FIG. 1 is a perspective view of an embodiment of a DMVA device adapted to be deployable onto a heart by a minimally invasive surgical procedure; FIG. 2A is a side cross-sectional view of the DMVA device 2000 of FIG. 1 comprising a liner having a single-wall central elastic region that forms an inflatable cavity in cooperation with the shell wall of the device, shown in a state of completing assistance of diastolic expansion of a heart, taken along the lines 2A/B-2A/B of FIG. 1; FIG. 2B is a side cross-sectional view of the DMVA device of FIG. 1 and FIG. 2A shown in a state of completing assistance of systolic compression of a heart, taken along the lines 2A/B-2A/B of FIG. 1; and FIG. 3 is an exploded perspective view of the DMVA device of FIG. 1.

Referring to FIGS. 1-3, DMVA device 2000 comprises a cup-shaped shell 2100, a cup-shaped support cage 2200 disposed within the cup-shaped shell, a liner 2300 forming an inflatable cavity for actuation of the ventricles of a heart, and a fitting 2500 for connecting to at least a first lumen (not shown) at the apex 2102 of cup 2000 so that the lumen is in fluid communication with the open inner volume 2009 of device 2000.

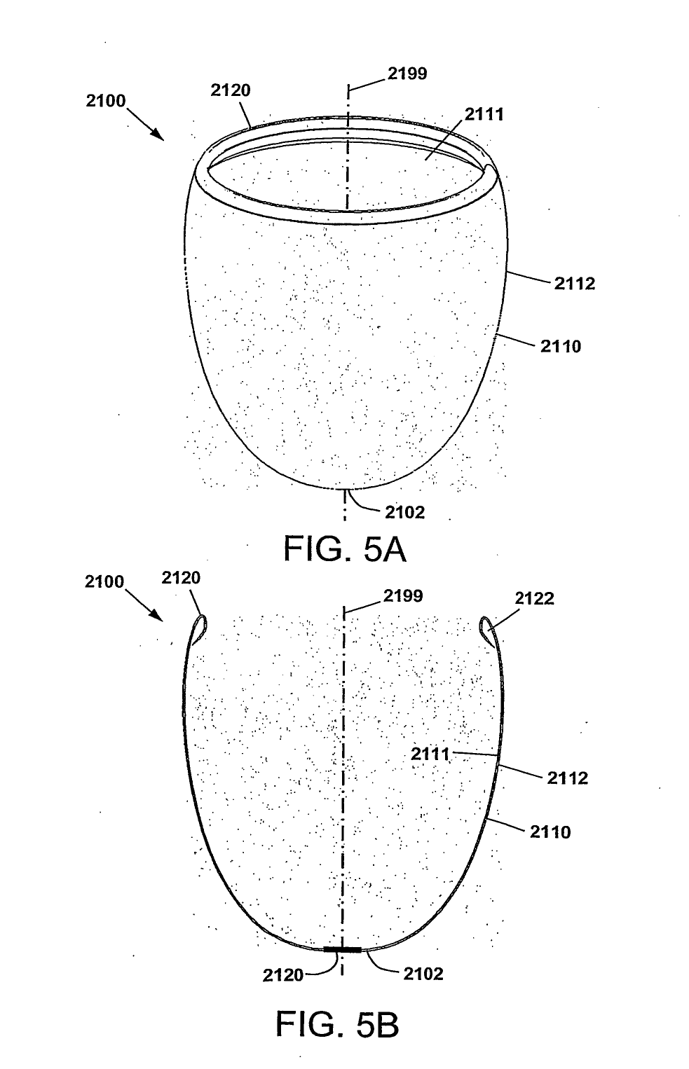

FIGS. 5A and 5B are perspective and side cross-sectional views of the shell of the DMVA device of FIGS. 1-3, respectively. Referring also to FIGS. 5A and 5B, cup shaped shell 2100 has an oblong shape oriented along a longitudinal axis 2199, and comprises a wall 2110 having an inner surface 2111 and an upper surface 2112 extending up to a rim 2120. Cup-shaped shell 2100 further comprises a hole 2120 at apex 2102 to enable the placement and passage through such hole 2120 of fluid connection fitting 2500, as will be explained subsequently in this specification.

In one embodiment, shell 2100 may be formed from a polymer-fiber composite. In one embodiment, shell 2100 consists essentially of DACRON™ polyester fiber and polyurethane polymer, wherein the polymer fiber is repeatedly wound substantially circumferentially, but at varying angles in the axial direction to form a fiber matrix of substantially uniform density. The wound fiber is saturated with the polyurethane resin and cured to form shell 2100 with a thin, flexible wall. Fabrication of such wound fiber-polymer forms is well known in the formation of various structures such as e.g., lightweight fluid vessels and fluid hoses. See for example, U.S. Pat. No. 6,190,481 of Yasushi et al., “Pressure vessel and process for producing the same;” U.S. Pat. No. 6,176,386 of Beukers et al., “Pressure-resistant vessel;” U.S. Pat. No. 4,220,496 of Carley, “High strength composite of resin, helically wound fibers and chopped fibers and method of its formation;” and U.S. Pat. No. 4,220,497 of Carley, “High strength composite of resin, helically wound fibers and swirled continuous fibers and method of its formation.” The disclosures of these United States patents are incorporated herein by reference.

In one embodiment, shell 2100 may be provided with a wall thickness of between about 0.01 and about 0.10 inches, and may further be from about 0.020 to about 0.060 inches. Wall 2110 of shell 2100 can thus be flexed and collapsed sufficiently to be contained in a minimally invasive deployment tool. However, when shell 2100 is expanded to its open state shown in FIGS. 1-5B, shell 2100 is resistant to further axial or circumferential expansion, i.e. shell 2100 is substantially isovolumetric with respect to expansion, by virtue of the non-expandable matrix of fiber and resin. Such isovolumetric property renders shell 2100 with the capability to be used as the outer volume constraint in a DMVA device. This capability will be described in further detail subsequently in this specification.

In other embodiments, cup shell 2100 may be formed with the fiber reinforcement portion of the polymer-fiber composite being of chopped fiber, or of a woven fiber mesh, i.e. a woven cloth.

Referring again to FIGS. 2A, 5A, and 5B, and in one embodiment depicted therein, shell 2100 further comprises an annular chamber 2122 formed at rim 2120. In one embodiment, a connection (not shown) to a lumen (not shown) is provided for the purpose of being able to inflate annular chamber 2122 after deployment of device 2000 on a heart, thereby providing greater structural strength at rim 2120, and the capability to change the shape of annular chamber 2122. At higher pressures, the aspect ratio of annulus height 2198 to annulus width 2197 decreases. Since the outer diameter 2196 of the annular chamber (or annulus) and the overall shell 2100 is constrained by the lack of elasticity of the polymer-fiber composite wall 2110, when annulus 2122 is inflated with a fluid, the inner diameter 2194 of annulus 2122 decreases, and the inner walls of annulus 2122 move inwardly toward central axis 2099 of apparatus 2000 as indicated by arrows 2195.

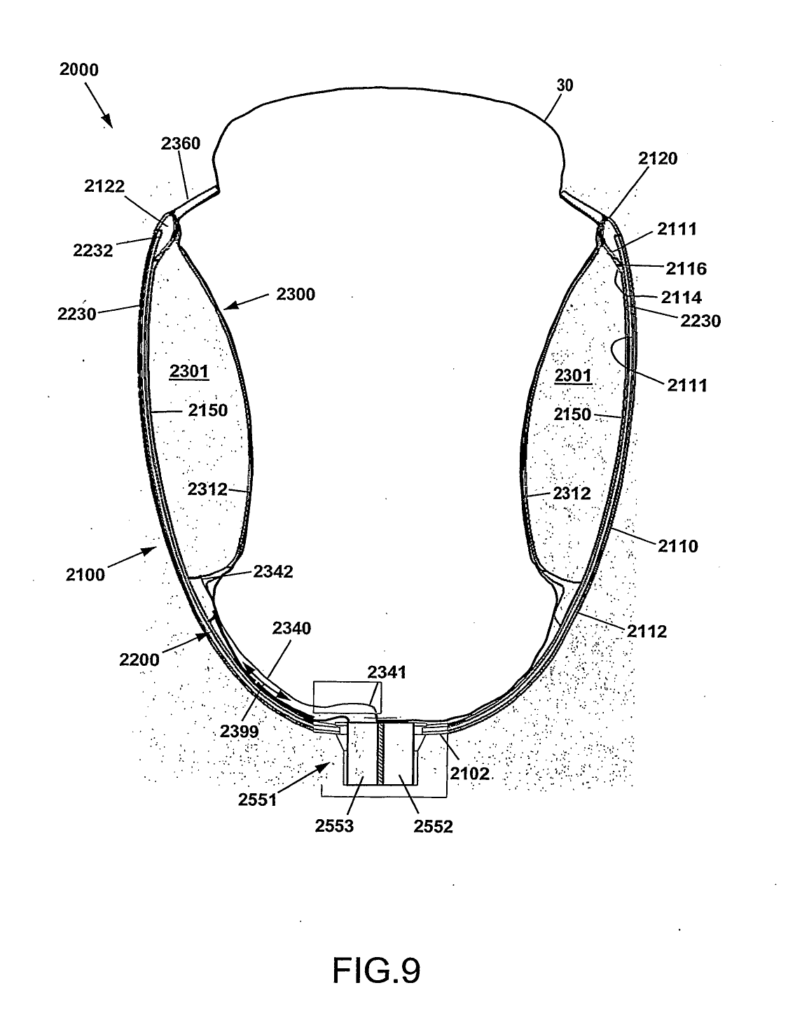

Such a motion of the inner walls of annulus 2122 provides a constricting effect at the atrio-ventricular (AV) groove of the heart 30 (see FIG. 9), thereby helping to retain apparatus 2000 secured to heart 30. Such a constricting effect may also reduce the overall diameter of a diseased heart at the AV groove; this reduction in diameter may in turn result in a reduction in the diameters of the tricuspid and/or mitral valves, thereby preventing blood regurgitation that may occur in diseased valves during systolic compression. In addition, changing the shape of the annular chamber 2122 by inflation also provides some adjustability of the angle 2193 of seal 2360 of liner/seal 2300, which is joined to shell 2100. As annular chamber 2122 is inflated and swells inwardly as indicated by arrows 2195, the lip 2362 of seal 2360 rotates upwardly and outwardly as indicated by arcuate arrow 2192.

Referring again to FIG. 2B and FIG. 5B, and in one embodiment depicted therein, annular chamber 2122 is formed by rolling the upper edge 2114 inwardly and downwardly, and joining the inner surface 2111 of wall 2110 to itself at bonding region 2116. Such a joining can be made by use of an adhesive at bonding region 2116. In FIGS. 2A, 2B, and 5B the length of bonding region is shown as being relatively small, and in some embodiments, may be as small as 0.01 inch. However, it is to be understood that upper edge 2114 of wall 2110 may extend downwardly a greater distance along inner surface 2111 of wall 2110 to provide a longer bonding region, on the order of about 0.25 to about 0.5 inch. It is also noted that struts 2230 of support cage 2200 also pass upwardly through bonding region 2116, and are joined to the inner surface 2111 of wall 2110 at bonding region 2116.

Although the non-expandable matrix of fiber and resin renders shell 2100 substantially isovolumetric with respect to expansion as noted previously, shell 2100 comprised of a thin wall of polymer-fiber composite does not by itself have a similar resistance to collapse under conditions of negative pressure, i.e. vacuum. In use, vacuum is applied to the inflatable cavity of the DMVA device 2000 to proactively evacuate such inflatable cavity, thereby providing assistance to diastolic filling of the heart ventricles. Thus, there can be a need for an additional support structure in the “minimally invasively deployed” DMVA devices described herein to prevent any substantial collapse of the shell 2100 thereof during diastolic assistance to the heart. A large amount of such collapse would render the DMVA device 2000 non-functional; a lesser degree of such collapse would reduce the volumetric efficiency of the device, and also result in wasted motion within the chest cavity of the patient, possibly irritating adjacent tissue, and causing discomfort to the patient.

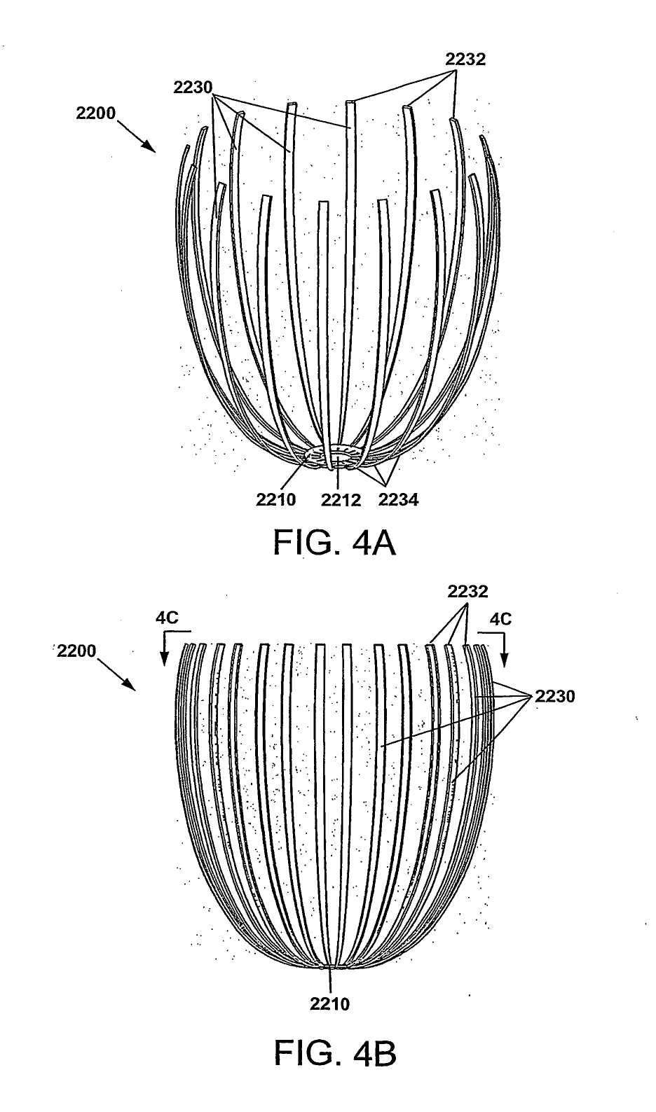

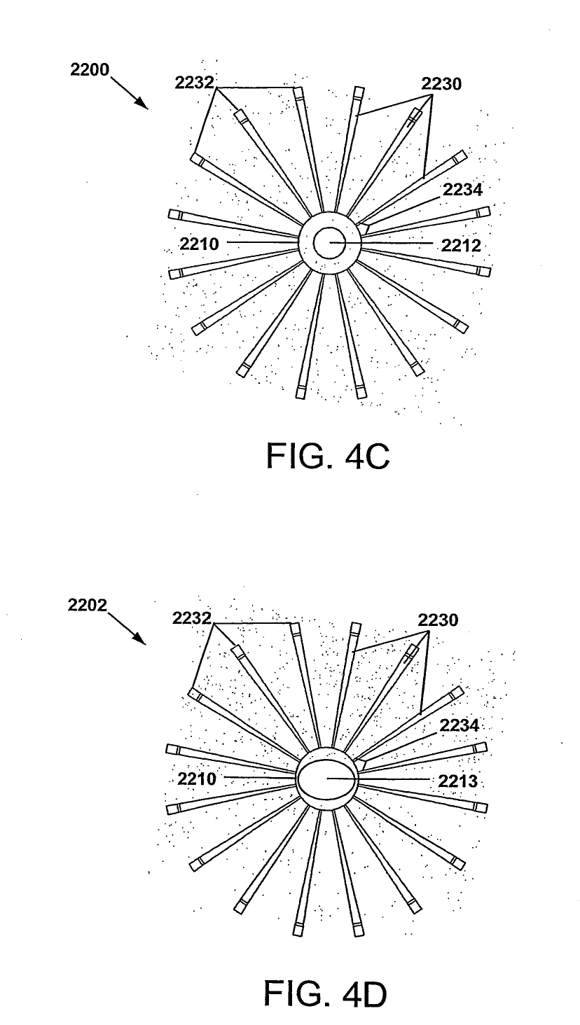

In one embodiment, such support is provided by a cup-shaped support cage that is joined to the inner surface of the cup shell 2100. FIG. 4A is a perspective view of the support cage of the DMVA device of FIGS. 1-3; FIG. 4B is a side elevation view of the support cage of FIG. 4A; and FIG. 4C is a top view of the support cage of FIGS. 4A and 4B, taken along line 4C-4C of FIG. 4B. Referring to FIGS. 2A-4C, support cage 2200 is comprised of a radial array of tines or struts 2230 extending outwardly and upwardly from a central disc 2210. In one embodiment depicted in FIGS. 4A-4C, support cage 2200 is comprised of an array of sixteen struts. It will be apparent that arrays of more or fewer struts may be used, and still provide the required support needed to prevent inward deformation of the cup shell wall 2110 during diastolic assistance to the heart, as described previously herein. In general, the number of struts may be between 8 and 24, with the exact number depending upon the flexibility and shape of such struts, and the flexibility of the wall 2110 of the cup shell 2100.

Referring again to FIGS. 2A-4C, and in one embodiment depicted therein, struts 2230 are provided with a tapered shape, such that struts 2230 are at a substantially maximum width at the tips 2232 thereof, and taper to a substantially minimum width at the innermost portion 2234 thereof. In one embodiment (not shown), struts 2230 may also taper in their respective radial planes along their lengths. In one embodiment, struts 2230 taper from a minimum thickness at their termini or tips 2232 to a maximum thickness at their innermost portions 2234. By providing such tapered cross-sections, the flexibility of struts 2230 may be made variable along their lengths, and “tuned” or matched to the particular support requirements along the wall 2110 of cup shell 2100.

Cage 2200 may be formed from any material that is sufficiently flexible so as to be able to be collapsed inwardly in a compact shape, so that when cage 2200 is made as a part of overall DMVA device 2000, such device 2000 may be collapsed and placed within a tube for subsequent deployment in a minimally invasive surgical procedure (to be described subsequently in this specification). For example, cage 2200 may be formed from a carbon fiber-polymer composite, a glass fiber-polymer composite, a flexible biocompatible polymer, or one or more metals and/or metal alloys typically used in forming small springs.

In one embodiment, cage 2200 is formed from a sheet of nitinol, a nickel-titanium shape memory alloy having a thickness of between about 0.015 and about 0.050 inches, and may further be between about 0.025 and about 0.035 inches. It will be apparent that the particular thickness of such material may vary considerably, depending upon the number of struts used in cage 2200, the width of the struts, and the properties of the material used in fabricating cage 2200.

In one embodiment, cage 2200 is first die stamped or cut using suitable means such as, e.g., laser cutting from a planar sheet of such steel to produce a flat “star-shaped” piece. (Not shown; similar in appearance to the view depicted in FIG. 4C.) Cage 2200 is then formed into the desired cage shape by a second die, and cage 2200 is then heat treated using well known spring steel production processes to provide the desired elastic modulus and resistance to yielding at the desired stress levels that occur when the DMVA device 2000 is packaged for minimally invasive deployment and when such device 2000 is in use in a patient. In other embodiments, cage 2200 may be formed by other processes such as wire forming, stamping, photo etching, chemical etching, and electroforming. Depending upon the particular process used, the cage 2200 may be initially formed in a planar geometry, and subsequently further formed into a cup shape by use of forming tooling. Alternatively, the individual struts 2230 may be formed separately and spot welded to circular disc 2210 to form the overall cup shaped structure of cage 2200.

Referring again in particular to FIGS. 4A and 4C, cage 2200 is further provided with a central hole 2212 in which fluid connection 2500 (see FIGS. 1, 2A, and/or 3) is fitted. When cage 2200 is installed in DMVA device 2000, central hole 2212 is coaxially aligned with hole 2120 of cup shell 2100, so that fluid connection 2500 is fitted within and sealingly engaged with hole 2110 of cup shell 2100 and hole 2212 of cage 2200.

The integration of cage 2200 into DMVA device 2000 will now be described, and is best understood with reference to FIGS. 2B, 3, and 4A. Referring to FIGS. 2B, 3, and 4A, cage support 2200 is placed within cup shell 2100 such that central hole 2212 of cage 2200 is coaxially aligned with hole 2110 of cup shell 2100, and such that struts 2230 are contiguous with inner wall 2111 of cup shell 2100. In one embodiment (not shown) cage 2200 is provided such that in its free unconstrained state, at any given horizontal cross section, cage 2200 is between about 10 percent and about 30 percent larger in diameter than the diameter of cup shell 2100. In this manner, when cage 2200 is inserted within cup shell 2100, cage 2200 is preloaded, and thus provides a baseline initial resistance to inward deflection of shell 2100 during diastolic assistance of the heart.

The struts 2230 of cage 2200 extend outwardly and upwardly from central disc 2210 contiguously along the inner wall 2111 of cup shell 2110 to their respective termini 2232. Such termini may extend at least partially into the region of cup shell 2100 where annular chamber 2122 is formed in order to provide outer support to annular chamber 2122. As described previously, after cage 2200 is fitted within cup shell 2100, annular chamber 2122 is formed by rolling the upper edge 2114 inwardly and downwardly, and joining the inner surface 2111 of wall 2110 to itself at bonding region 2116. Termini 2232 may be provided with an engagement feature such as a T-shape for improved bonding to the cup shell 2100.

In one embodiment, after cage 2200 is fitted within cup shell 2100, the interior space 2009 of DMVA device is coated with a conformal and biocompatible coating 2150 which seals and further bonds cage 2200 to the inner surface 2110 of cup shell 2100. Coating 2150 also provides protection from direct contact between the inner surface 2312 of elastic wall 2310 of liner 2300 and struts 2320 of cage 2200, in the event that struts 2230 have sharp edges and could otherwise wear, abrade, or cut wall 2310 of liner 2300. Coating 2150 may consist essentially of a suitable coating that is biocompatible, has good adhesion to the inner shell wall 2111 and to struts 2230, and is sufficiently flexible so as to allow collapsing of the DMVA device 2000 into a tube without cracking. Examples of suitable coatings are ethylene propylene diene monomer (EPDM) rubber, Silastic, and materials used in biocompatible glues, such as fibrin glue.

Depending upon the selection of materials, coating 2150 may also have additional functions similar to those recited previously in the specifications of the aforementioned U.S. patent application Ser. Nos. 10/607,434, and 10/795,098. In one embodiment, coating 2150, or the lower region 2152 thereof (see FIG. 2A) that is in direct contact with the heart 30 (see FIG. 9) contains a therapeutic agent which is delivered directly to the heart 30. The selection and delivery of such therapeutic agents has been described previously in detail in the specifications of application Ser. Nos. 10/607,434, and 10/795,098. In another embodiment, coating 2150, or the lower region 2152 thereof (see also FIG. 2A), is a coating of a biocompatible thin film that facilitates ingrowth and adhesion of tissue. In this manner, after some period of use, the heart 30 becomes adhered to such coating in region 2152, thereby lessening or eliminating the need for the application of vacuum at port 2502 of fluid connection 2500.

For example, such coating may be comprised of fibronectin. In the Online Macromolecular Museum article by M. Ward et al., “Fibronectin, an Extracellular Adhesion Molecule” at http://www.callutheran.edu/BioDev/omm/fibro/fibro.htm, there is disclosed, “Fibronectin (FN) is involved in many cellular processes, including tissue repair, embryogenesis, blood clotting, and cell migration/adhesion. Fibronectin exists in two main forms: 1) as an insoluble glycoprotein dimer that serves as a linker in the ECM (extracellular matrix), and; 2) as a soluble disulphide linked dimer found in the plasma (plasma FN). The plasma form is synthesized by hepatocytes, and the ECM form is made by fibroblasts, chondrocytes, endothelial cells, macrophages, as well as certain epithelial cells.”

Such a coating may further include collagen. The surface of the coating may be “velour-like,” i.e., highly textured such that a large surface area is provided to promote ingrowth and adhesion of tissue.

The integration of liner 2300 into DMVA device 2000 will now be described, and is best understood with reference to FIGS. 2A, 2B, and 3. Referring to FIGS. 2A, 2B, and 3, liner 2300 is comprised of an upper bond region 2320, a central elastic wall region 2310, and a lower bond region 2330. In one embodiment, liner 2300 further comprises integral seal 2360, the function of which in sealing to the heart 30 has been described previously in the aforementioned U.S. patent application Ser. Nos. 10/607,434, and 10/795,098. It will be apparent that seal 2360 could be provided separately and suitably joined to the upper rim 2120 of cup shell 2100.

Although in FIGS. 2A and 2B liner 2300 is depicted as having a relatively thick lower rim 2332 for joining to shell 2100 at bonding region 2330, it is to be understood that rim 2332 may be provided proportionately less thick than as illustrated in FIGS. 2A and 2B, and still provide satisfactory results. Referring in particular to FIG. 2B, rim 2332 is joined to elastic wall region by a tapered transition section 2334 that is provided to minimize stress on the liner 2300 in the transition between the elastic wall region 2310 and the lower bonding region 2330. In like manner, a tapered transition section 2336 is provided in liner 2300 in proximity to upper bonding region 2320. Further details on these tapered transition sections in liners of the DMVA devices of the present invention have been described further in the aforementioned U.S. patent application Ser. Nos. 10/607,434, and 10/795,098. In other embodiments, liner 2300 may be provided with an elastic wall region and transition sections which are formed to provide a rolling diaphragm structure as described previously in the specification of the aforementioned U.S. patent application Ser. No. 10/607,434 and shown in FIGS. 4A-4C of the drawings of such application.

Liner 2300 is placed within the assembly comprising cup shell 2100, cage support 2200, and optionally, conformal coating 2150. Liner 2300 is joined to the inner surface of this assembly, in one embodiment by a suitable adhesive, at upper bond region 2320, and a lower bond region 2330. Thus, with liner 2300 joined and sealed to conformal coating 2150 at these bond regions, the central elastic wall region 2310 may be expanded inwardly by the delivery of fluid into cavity 2301 as illustrated in FIG. 2A to provide systolic assistance to heart 30 (see also FIG. 9), and the central elastic wall region 2310 may be retracted outwardly by the withdrawal of fluid from cavity 2301 as illustrated in FIG. 2B to provide diastolic assistance to heart 30.

In one embodiment, liner 2300 is made of a silicone polymer known commercially as Silastic, or Liquid Silicone Rubber and is provided in liquid form prior to curing to a solid form for use in the DMVA device 2000. One example of a material suited for liner 2300 is MED4850 Liquid Silicone Rubber. One example of an adhesive well suited for bonding elements consisting essentially of this material is MED1-4213. Both of these materials are products of the NuSil Technology Company, of Carpenteria, Calif.

Liner 2300 can be made of an elastomer having a Shore A durometer of between about 20 to about 70, and an elongation at break of at least about 200 percent, and may further be at least about 600 percent. In one embodiment in which liner 2300 is formed from liquid silicone rubber, liner 2300 has an elongation at break of about 900 percent.

In an alternative embodiment (not shown), liner 2300 is formed with a double wall, such that cavity 2301 is contained entirely within such liner. In this embodiment, liner 2300 comprises an inner elastic wall 2310 that is in contact with the heart and provides systolic and diastolic assistance as described previously. Liner 2300 further comprises an outer wall that is contiguously disposed along the corresponding central region of conformal coating 2150. In this embodiment, liner 2300 may be bonded at the upper and lower bonding regions as described previously, and at least at a portion of the centrally disposed outer wall of liner 2300.

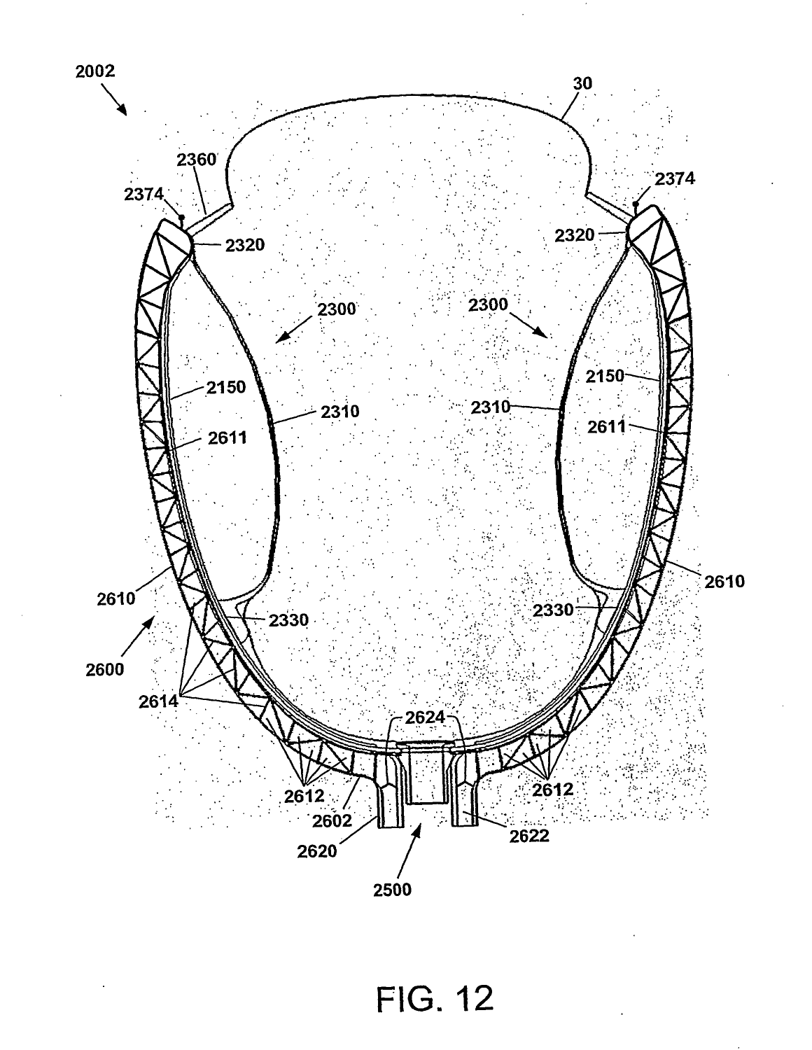

In a further embodiment, the inner elastic wall 2310 of liner 2300 is comprised of a multi-layer membrane which may contain therapeutic agents for delivery to the heart. Such a multilayer liner has been described previously in the specification of the aforementioned U.S. patent application Ser. No. 10/607,434, with reference in particular to FIG. 12 of the drawings of such application.

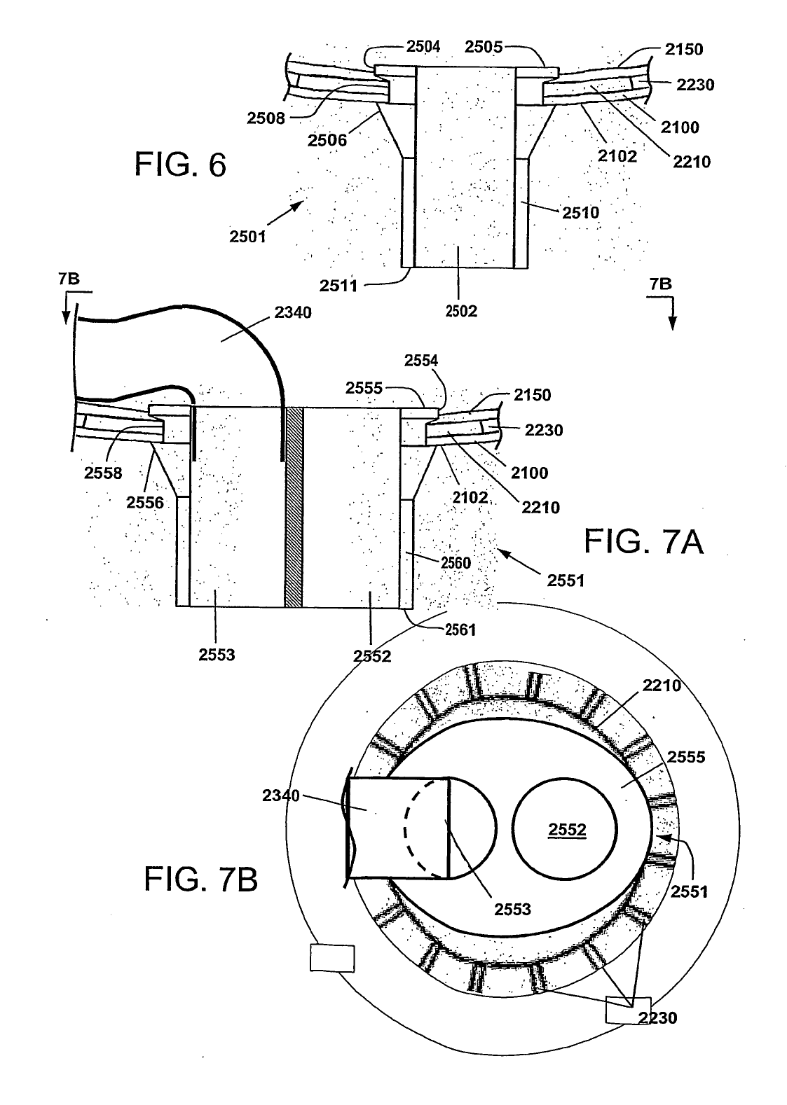

The fitting for connection of lumens to the DMVA device 2000 for the application of vacuum to the apex of the heart and for the delivery and withdrawal of drive fluid to cavity 2301 will now be described. FIG. 6 is a detailed cross-sectional view of one embodiment of a single port fitting 2501 for connecting to a first lumen at the apex of the DMVA apparatus shell 2100 to render the first lumen in fluid communication with the inner volume of the apparatus. Referring to FIG. 6, fitting 2501 is shown installed at the apex 2102 of device 2000 (see FIG. 1).

Fitting 2501 comprises an upper flange 2504, a lower flange 2506, and a recess 2508 between upper flange 2504 and lower flange 2506. Fitting 2501 further comprises an open port or passageway 2502 extending therethrough, from the top surface 2505 of upper flange 2504 to the lower surface 2511 of tubular body 2510. The upper and lower flanges 2504 and 2506 and the recess 2508 may be formed so as to be a snap fit within hole 2212 (see FIG. 4C) within disc 2210 of cage 2200, and hole 2120 (see FIG. 5B) of shell 2100. Other alternative structures for installing and sealing a fitting within a thin walled body are well known, and may include e.g. multi-piece threaded fittings, such as a bulkhead fitting. In one embodiment, after fitting 2501 is fitted to cup shell 2100 and cage 2200, coating 2150 is applied to the interior of device 2000, providing further sealing and securing of fitting 2501 therein. Although coating 2150 is shown terminating at the edge of upper flange 2504 of fitting 2501 in FIG. 6, it is to be understood that coating 2150 may partially or fully cover upper surface 2505 of fitting 2501.

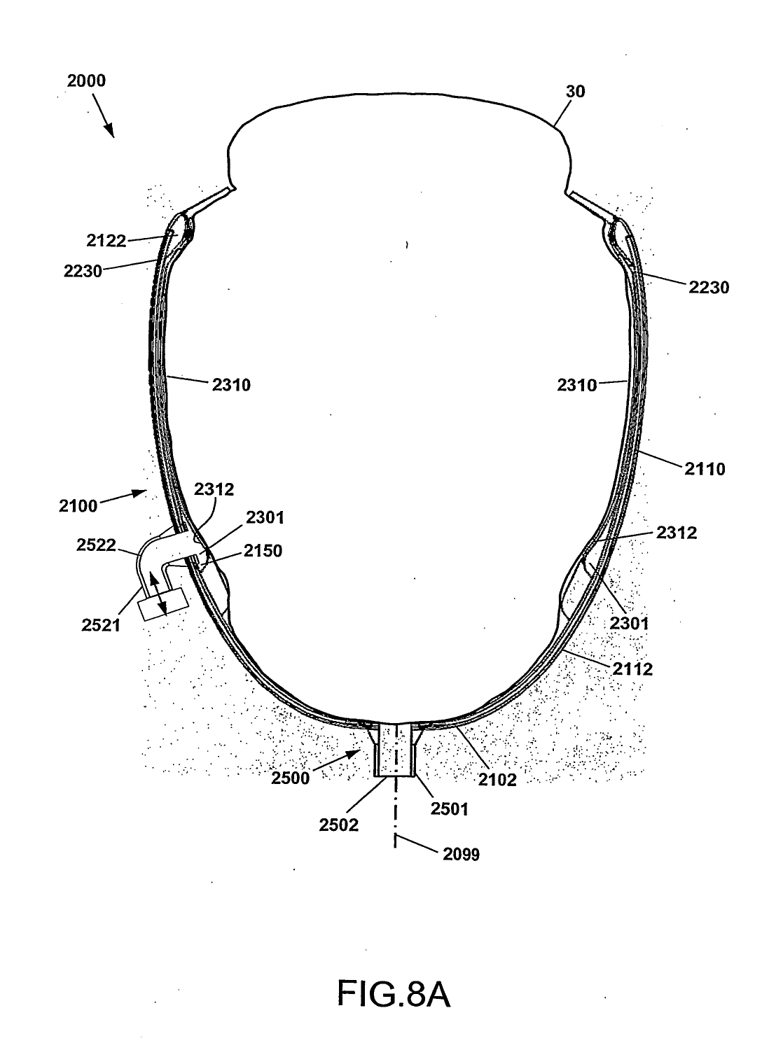

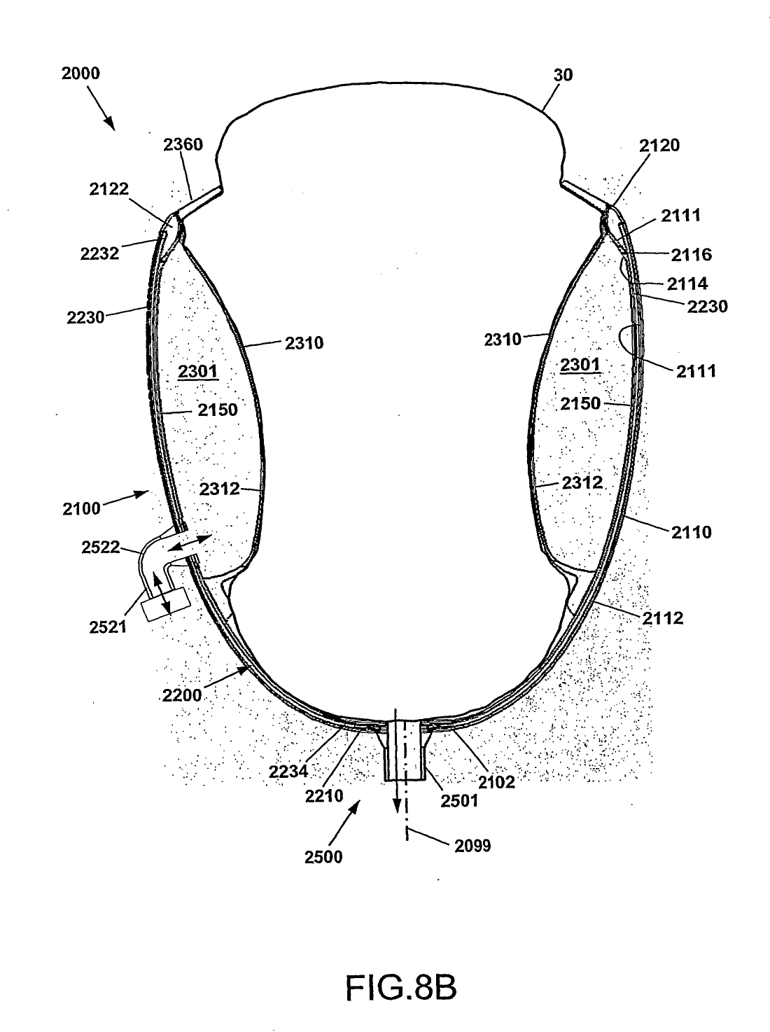

Referring to FIGS. 2A, 2B, 8A, and 8B, in like manner, a second single port fitting 2521 is provided in device 2000 for the delivery and withdrawal of DMVA drive fluid into cavity 2301 during operation. In one embodiment, fitting 2521 is similar in construction to fitting 2501 in the manner in which such fitting is joined to the wall 2110 of shell 2100, and sealed thereto by coating 2150, or by additional adhesive (not shown). In one embodiment, fitting 2521 is formed from a flexible elastomer, so that fitting 2521 may be deformed during the packaging of device 2000 in a minimally invasive tool (to be described subsequently in this specification), and during deployment in a minimally invasive procedure. Fitting 2521 may also be provided with an elbow 2522 integrally formed therein, such that the lumen attached thereto (not shown) is directed toward the general area of the apex 2102 of DMVA device 2000.

In one embodiment, (not shown) a second fitting is provided for supply and withdrawal of DMVA drive fluid into cavity 2301, in order to provide adequate flow capacity to and from cavity 2301. Such a fitting may be provided on the opposite side of device 2000, i.e., at 180 degrees around DMVA device 2000 from fitting 2521.

When device 2000 is installed in a patient and is in use, a first lumen (not shown) is connected to the body 2510 of fitting 2501 and vacuum is applied to heart 30 (see FIG. 8A) through the first lumen (not shown) connected to fitting 2501. For the sake of simplicity of illustration, a simple cylindrical tubular body 2510 is depicted in FIG. 6 for connection to a lumen. Many other suitable connection features may be provided in body 2510 such as e.g., a tubing barb, a threaded fitting, a bayonet fitting, a quick-connect fitting, or a LUER LOK™ fitting. In like manner, a second lumen (not shown) is connected to the body of fitting 2521, and DMVA drive fluid is supplied and withdrawn from cavity 2301 during systolic compression and diastolic expansion of the heart 30. In a further embodiment (not shown) such lumens may be integrally formed with their respective fittings and provided as part of the overall DMVA device 2000.

FIG. 8A is a cross-sectional view of the DMVA device 2000 of FIGS. 2A and 2B, shown assisting a heart at the completion of diastole, and FIG. 8B is a cross-sectional view of the DMVA device 2000 of FIGS. 2A and 2B shown assisting a heart at the completion of systole. Referring first to FIG. 8B, it can be further seen that the drive fluid has been fully delivered into cavity 2301 through fitting 2521, and that the outer surface of the ventricular region of the heart 30 is in close contact with elastic wall 2310 of liner 2300, resulting in full systolic compression of heart 30. Referring now to FIG. 8A, it can be seen that the drive fluid has been fully evacuated from cavity 2301 through fitting 2521, and that the outer surface of the ventricular region of the heart 30 remains in close contact with elastic wall 2310 of liner 2300, resulting in full diastolic expansion of heart 30.

In one embodiment, DMVA device 2000 is provided with a fitting for connecting to more than one lumen at the apex of the cup shell. FIG. 7A is a detailed side cross-sectional view of one embodiment of a unitary fitting for connecting to a first lumen at the apex of the DMVA apparatus shell 2100 to render the first lumen in fluid communication with the inner volume of the apparatus and for connecting to a second lumen to render the second lumen in fluid communication with an inflatable cavity in an elastic central region of the liner. FIG. 7B is an axial view of one embodiment of the unitary fitting of FIG. 7A, taken along line 7B-7B of FIG. 7A. FIG. 4D is a top view of a support cage similar to the cage of FIGS. 4A-4C, but provided with an elliptical central hole for engagement with the unitary fluid fitting of FIGS. 7A and 7B.

Referring to FIGS. 7A, 7B, and 4D, fitting 2551 is similar to fitting 2501 of FIG. 6 in structure, and in the manner in which such fitting is fitted in DMVA device 2000. Fitting 2551 is generally oblong or elliptical in cross section, and comprises an upper flange 2554, a lower flange 2556, and a recess 2558 between upper flange 2554 and lower flange 2556. Fitting 2551 further comprises a first open port or passageway 2552 and a second open port or passageway 2553 extending therethrough, from the top surface 2555 of upper flange 2554 to the lower surface 2561 of tubular body 2560. The upper and lower flanges 2554 and 2556 and the recess 2558 may be formed so as to be a snap fit within hole 2213 within disc 2210 of cage 2202, and hole 2120 (see FIG. 5B) of shell 2100.

When DMVA device 2000 is installed in a patient and is in use, a first lumen (not shown) is connected to port 2552 of fitting 2551 and vacuum is applied to heart 30 (see FIG. 9) through such first lumen connected to fitting 2501. Additionally, a second lumen (not shown) is connected to port 2553 of fitting 2551 for the delivery and withdrawal of drive fluid through such second lumen and through third lumen or passageway 2340 disposed within device 2000 and connected to cavity 2301 (see FIGS. 2A and 2B). In one embodiment (not shown), the first (vacuum) lumen and the second (drive fluid) lumen are formed as a single unitary tubular structure, in order to simplify the connection to DMVA device 2000 during installation in the patient.

FIG. 9 is a cross-sectional view of a DMVA device similar to the device of FIGS. 1-3 but fitted with the dual port fitting of FIGS. 7A and 7B, shown assisting a heart at the completion of systole. Referring to FIG. 9, it can be seen that internal lumen 2340 is disposed inside of device 2000 near the apex 2102 thereof, between conformal coating 2150 and the apical region of heart 30. Internal lumen 2340 is operatively connected at proximal end 2341 to port 2553 of fitting 2551, and at distal end 2342 to cavity 2301 formed between liner 2300 and shell 2100, cage 2200, and/or coating 2150. During operation, drive fluid is delivered into cavity 2301 and withdrawn from cavity 2301 as indicated by bidirectional arrow 2399; and vacuum is applied to the apex of heart 30 through port 2552 of fitting 2551. In FIG. 9, internal lumen 2340 is shown as having a relatively small cross-section as compared to the cross section of port 2553. In order to provide sufficient flow capacity, lumen 2340 may have a “flattened” oblong elliptical or rectangular shape to meet the required flow capacity, or a plurality of lumens or liner passageways may connect port 2553 to cavity 2301.

In another embodiment (not shown), a multiport fitting is provided in which vacuum may be applied through a first central passageway, and DMVA drive fluid may be delivered or withdrawn through a second concentric passageway. The second concentric passageway is connected to a plurality of radially distributed “spoke” passageways formed or embedded in the shell wall 2110, such passageways being in communication with cavity 2301 of DMVA device 2000.

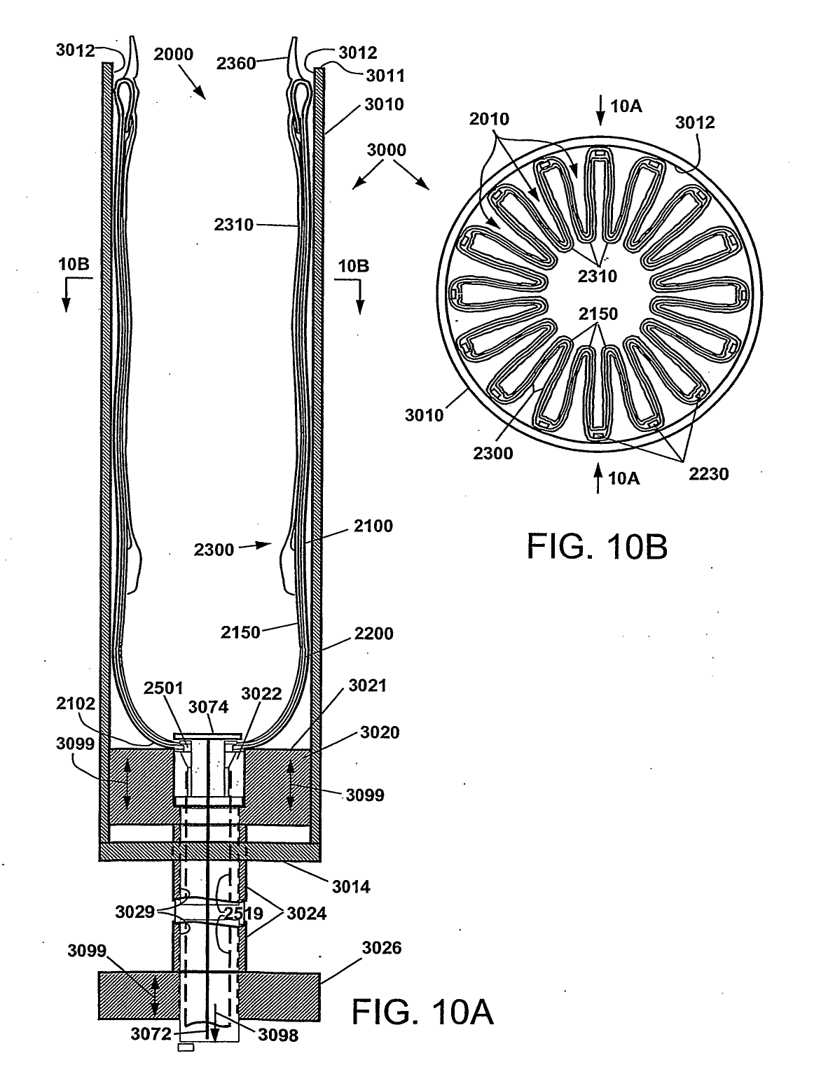

FIG. 10A is a longitudinal cross-sectional view of the DMVA device 2000 of FIGS. 1-3 shown collapsed and inserted into an endoscopic tool 3000 for deployment onto a heart. FIG. 10B is an axial cross-sectional view of the DMVA device 2000 and tool 3000 of FIG. 10A, taken along the line 10B-10B of FIG. 10A. This tool accomplishes a similar function to that of a tool for deployment of a cardiac mesh harness, as disclosed in United States Patent Application Publication 2005/003322 A1, the disclosure of which is incorporated herein by reference. Referring to FIGS. 10A and 10B, the DMVA device 2000 of FIGS. 1-7B is shown as being collapsed and inserted into tube 3010 of deployment tool 3000.

Deployment tool 3000 comprises an elongated tube 3010 within which piston 3020 is slidably disposed. Piston 3020 is operatively connected at the lower surface thereof to piston rod or plunger 3024, which can include end knob 3026. Plunger 3024 is bidirectionally movable and slidable through end plate 3014 of tube 3010, resulting in piston 3020 being bidirectionally movable within bore 3012 of tube 3010, as indicated by arrows 3099. For the sake of space limitations in FIG. 10A, a substantial section of plunger 3024 is not shown, with it being understood that plunger 3024 is of sufficient length to displace piston 3020 to the distal end of tube 3010 during a procedure to deploy DMVA device 2000 onto a heart.

When using tool 3000 in a minimally invasive procedure for deploying DMVA device 2000 onto a heart, DMVA device 2000 is collapsed and inserted into bore 3012 of tube 3010. Access to the heart can be obtained through incisions in the thorax and pericardium, and the distal end 3011 of tube 3010 is then placed through such incisions in the pericardium at the apex of the heart. Suitable surgical access procedures include a sub-xyphoid incision and approach, a subcostal incision and approach, or a mini-thoracotomy, optionally including ultrasound or video-assisted thorascopic imaging and guidance.

Plunger 3024 is operated by the surgeon such that DMVA device 2000 is pushed out of bore 3012 of tube 3010 of tool 3000. As DMVA device 2000 is deployed from tool 3000, the struts 2232 of the cage 2200 of DMVA device 2000 transition from their collapsed shape within tube 3010 to their deployed shape as illustrated in FIGS. 2A-4B. DMVA device 2000 thus opens to its operational state depicted in FIGS. 1-2B and envelops the heart as DMVA device 2000 is deployed from tool 3000.

Referring again to FIG. 10A, and in one embodiment, piston 3020 of tool 3000 is provided with a cavity 3022 formed in the outer surface 3021 thereof, for the purpose of receiving fitting 2501 of DMVA device 2000 (or fitting 2551 of FIGS. 7A and 7B). In this manner, DMVA device 2000 is deployed from tool 3000 with the deployment force being borne by cup shell 2100 and cage support 2200. If such a cavity is not provided, stress on fitting 2501 during deployment may adversely affect the integrity of the seal of fitting 2501 to shell 2100 and cage support 2200.

In another embodiment, piston 3020, plunger 3024, and knob 3040 are provided with a bore 3029 extending from cavity 3022 of piston 3020 through knob 3040. A temporary lumen 2519 may be connected to fitting 2501 to apply vacuum to device 2000 contained in tool 3000, resulting in vacuum assistance which helps “pull” device 2000 onto heart 30 during deployment. Alternatively, both the vacuum lumen (not shown) and the DMVA drive fluid lumen (not shown) may be connected to the appropriate respective ports on DMVA device 2000, or may be formed as an integral part thereof, and such lumens may be fed through bore 3029 during deployment of device 2000. When DMVA device 2000 is fitted to heart 30, tool 3000 is removed from the chest cavity of the patient, and the lumens slide out through bore 3029 of tool 3000, and are left in place ready for connection to the DMVA device vacuum and drive fluid sources.

In the cross-sectional view of deployment tool 3000 and DMVA device 2000 depicted in FIG. 10A, for the sake of simplicity of illustration, such cross-section has been illustrated showing only a “slice” or a single plane taken through the central axis of tool 3000 and DMVA device 2000, at line 10A-10A of FIG. 10B. In this single plane cross-sectional view at plane 10A-10A of FIG. 10B, the shell 2100, cage support 2200, liner 2300 including seal 2360, and fitting 2501 of device 2000 can be seen. The struts 2230 have flexed substantially near the apex 2102 of cup shell 2100, and are disposed in proximity to the inner wall of bore 3012 of tube 3010.

Referring also to FIG. 10B, the highly flexible cup shell 2100, conformal coating 2150, and liner 2300 are arrayed in a generally folded arrangement between the struts 2360. It will be apparent that the exact arrangement of the struts 2230 and folds 2010 of DMVA device 2000 within tube 3010 will vary with a number of factors, including the number of struts 2230 provided in the cage support 2200, the materials and thicknesses of liner 2300, conformal coating 2150, and shell 2100, and the manner in which the device is collapsed and placed within deployment tube 3010. In one embodiment, the arrangement of folds 2010 may be less orderly and symmetric than is depicted in FIG. 10B, with some portion of the struts displaced inwardly toward the center of tube 3010, while others are disposed outwardly proximate to the inner wall of bore 3012 of tube 3010. It is to be understood that the manner of packing of DMVA device 2000 in tool 3000 depicted in FIGS. 10A and 10B is for illustrative purposes only, and that other folding arrangements of device DMVA 2000 within tool 3000 can be used to attain satisfactory results during a subsequent deployment procedure.

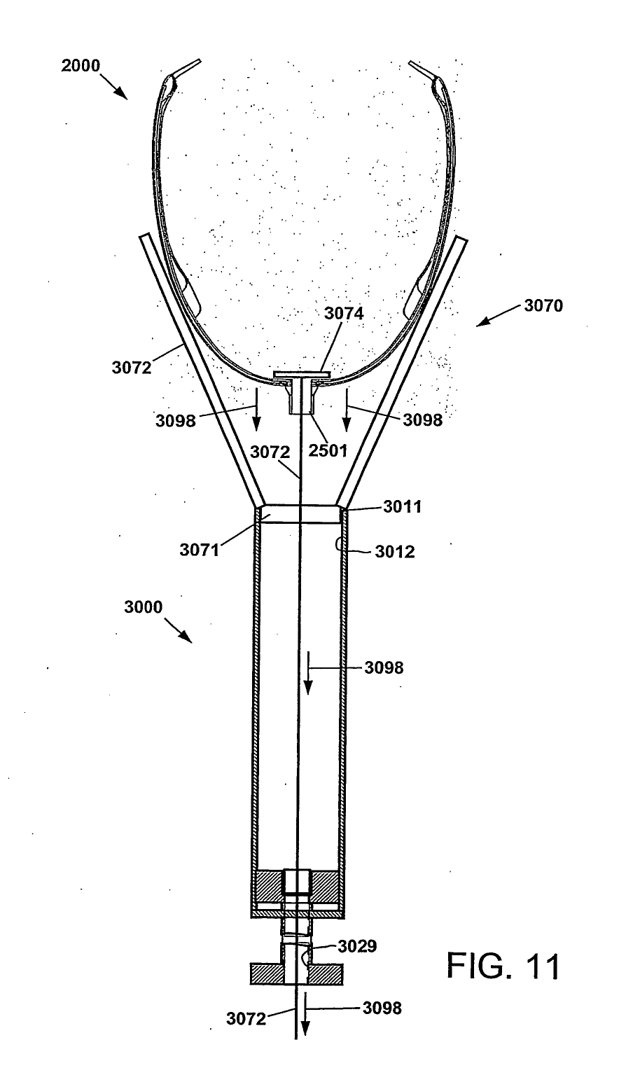

FIG. 11 is a cross-sectional view of an embodiment of a tool provided for use in loading DMVA device 2000 into deployment tool 3000 of FIG. 10A. Referring to FIG. 10A and FIG. 11, DMVA device 2000 is collapsed by pulling such device inwardly through a tapered funnel 3070 that is disposed at the distal end 3011 of deployment tool 3000. Funnel 3070 is generally conical in shape and may be further provided with a short neck section 3071, which fits into bore 3012 of tool 3000 with a mild interference fit. Such a snug fit of funnel 3070 into tool 3000 provides stability of the funnel and tool assembly during the pulling of DMVA device 2000 into tool 3000.

In one embodiment, such pulling action is accomplished by the provision of a guide wire 3072, which is attached at the distal end thereof to a disc 3074 placed on the inside of fitting 2501 of DMVA device 2000. Such guide wire 3072 is fed through the bore 3012 of tool 3000. When guide wire 3072 is pulled as indicated by arrow 3098, DMVA device 2000 is drawn into funnel 3070, thereby collapsing device 2000 and pulling DMVA device 2000 into the bore 3012 of tool 3000 as shown in FIG. 10A.

In one embodiment (not shown), funnel 3070 may be a fluted funnel, and may have the same number of flutes as DMVA device 2000 has struts 2230 (see FIG. 10B). This feature on funnel 3070 results in a more structured and uniform collapse of DMVA device 2000, resulting in an orderly folding of the shell 2100 of DMVA device 2000, as depicted in FIG. 10B.

In another embodiment (not shown), instead of using guide wire 3072 connected to disc 3074, a hook shaped device is extended through passageway 3029 of tool 3000 and through fitting 2501 of DMVA device 2000, and engaged with the inside of fitting 2501. Such hook device is then used to collapse and draw device 2000 into the bore 3012 of tool 3000.

Alternatively or additionally, a strutted collapsing device (not shown) may be provided similar to the support mechanism in an umbrella, wherein the struts are expandable so as to be interspersed between the struts 2230 and substantially parallel to the upper portions of such struts of DMVA device 2000. The struts are then contracted inwardly in the radial direction thus collapsing DMVA device 2000 into the configuration depicted in FIGS. 10A and 10B. In one embodiment, the number of struts of the collapsing device is equal to the number of struts 2230 of device 2000, and each of the struts of the collapsing device forms a fold in device 2000, such folds being substantially the same as folds 2010 in FIG. 10B. With DMVA device 2000 rendered in a collapsed configuration, DMVA device 2000 may be inserted into the bore 3012 of tool 3000. The strutted collapsing tool then releases device DMVA 2000, which remains in the collapsed configuration as shown in FIGS. 10A and 10B, while the collapsing tool is withdrawn.

Alternatively or additionally, a vacuum source may be connected to deployment tool 3000, such as through passageway 3029. By providing a vacuum within bore 3012 of tool 3000, vacuum assistance may be used to collapse and draw DMVA device 2000 into tool 3000.

Referring again to FIGS. 10A and 10B, deployment tool 3000 is made of biocompatible materials that are suitable for surgical procedures. In one embodiment, deployment tool 3000 is made of a suitable surgical stainless steel such as T304 stainless steel, or 316L stainless steel. In other embodiments, deployment tool may be made of relatively inexpensive plastics such as polyvinyl chloride (PVC), and be intended for use as a disposable unit. In a further embodiment (not shown), deployment tool is comprised of absorbent material, in order to provide the capability of wicking up bodily fluids during deployment. Such capability enables the surgeon and assisting staff an unobstructed view of the device and bodily tissues during the installation procedure.

In one embodiment, the tube 3010 of tool 3000 has an outside diameter of about two inches, and a bore 3012 (inner) diameter of about 1.95 inches. In other embodiments, the tube 3010 of tool 3000 can have an outside diameter of about 1.5 inches, or even about 1 inch. In instances where the DMVA device 2000 is sized for pediatric use, tube 3010 has an outside diameter of about 1 centimeter. For a full adult-sized DMVA device 2000, tube 3010 is about 8 inches in overall length, so that a total stoke length of piston 3020 of about 7 inches is provided, in order for the tool 3000 to fully discharge device 2000 from the bore 3012 thereof during a minimally invasive installation procedure on a patient. In like manner, the length of rod 3024 is also provided at about 7 inches to render the required piston stroke length. It will be apparent that the sizes of the components of tool 3000 will vary somewhat, in order to match the various sizes in which DMVA device 2000 is provided.

Referring again to FIG. 10A, tool 3000 is depicted as comprising a simple short cylindrical gripping knob 3040. It will be apparent that a variety of other knob shapes may be suitable. In particular, knobs with ergonomic features such as recesses, forms, and/or textured surfaces to match, engage and/or provide traction with the surgeon's fingers are desirable.

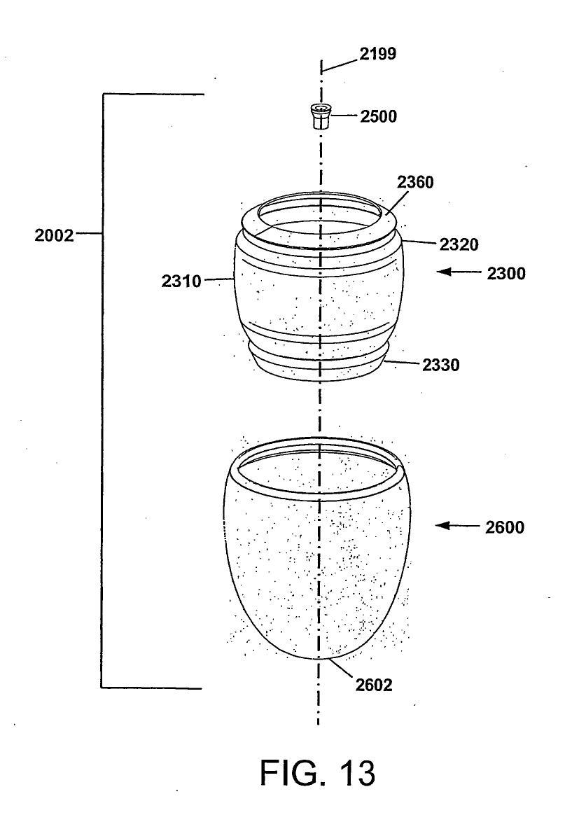

FIG. 12 is a side cross-sectional view of another embodiment of a DMVA device that is capable of being deployed onto a heart by a minimally invasive surgical procedure, shown in a state of completing assistance of systolic compression of a heart. FIG. 13 is an exploded perspective view of the embodiment of FIG. 12. Referring to FIGS. 12 and 13, DMVA device 2002 is similar to the DMVA device 2000 of FIGS. 1-9, with the main difference being in the structural shell 2600 of DMVA device 2002. Structural shell 2600 of DMVA device 2002 is used in lieu of shell 2100 and cage support 2200 of DMVA device 2000 of FIGS. 1-9.

DMVA device 2002 comprises a cup-shaped shell 2600, a liner 2300 forming an inflatable cavity with the inner surface 2611 of wall 2610 of shell 2600 for actuation of the ventricles of a heart 30, and 2500 for connecting to at least a first lumen (not shown) at the apex 2602 of cup 2002 so that the lumen is in fluid communication with the open inner volume of device 2002. Structural shell 2600 provides the overall support and external spatial constraint to liner 2300, such that when DMVA drive fluid is delivered into and withdrawn from cavity 2301, elastic liner wall region 2310 of liner 2300 expands inwardly as shown in FIG. 12, and recoils back, thereby providing systolic and diastolic assistance to heart 30 as described previously.

As was described for DMVA device 2000 of FIGS. 1-9, liner 2300 is comprised of an upper bond region 2320, a central elastic wall region 2310, and a lower bond region 2330. Liner 2300 is joined to the inner surface 2611 of shell 2600, such as by a suitable adhesive, at upper bond region 2320, and a lower bond region 2330. In one embodiment, a coating 2150 is provided on inner surface 2611 of shell 2600 to improve adhesion at bond regions 2320 and 2330.

Shell 2600 is collapsible and inflatable, and prior to deployment onto a heart, DMVA device 2002 including shell 2600 and liner 2300 is in a collapsed state. Referring again to FIG. 12, shell 2600 may be inflated from a collapsed state to an inflated state by the temporary connection of a pressurized fluid supply to annular fitting 2620. An inflating fluid is delivered inwardly through annular fitting 2620, and flows upwardly through a matrix of interstitial compartments 2612, which are interconnected via internal passageways (not shown). Such inflating fluid may be the Silastic liquid silicone rubber materials previously describe herein, or a liquid foam that fills interstitial compartments 2612 and subsequently cures. Such inflating fluid may be a multiphase fluid containing solid particles and/or elongated fibers, such that a reinforcing composite material is formed in interstitial compartments 2612.

In a further embodiment, one or more of the materials used in forming shell 2600 may contain radiopaque material that shields the heart from radiation during certain medical imaging procedures.

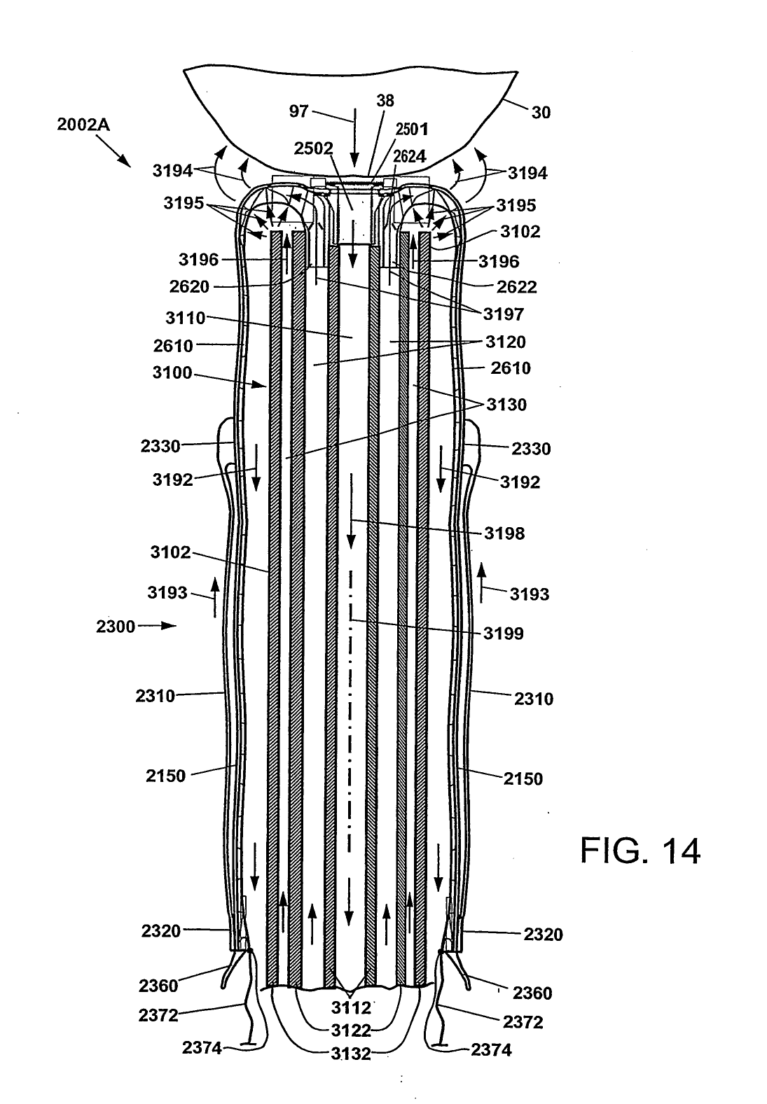

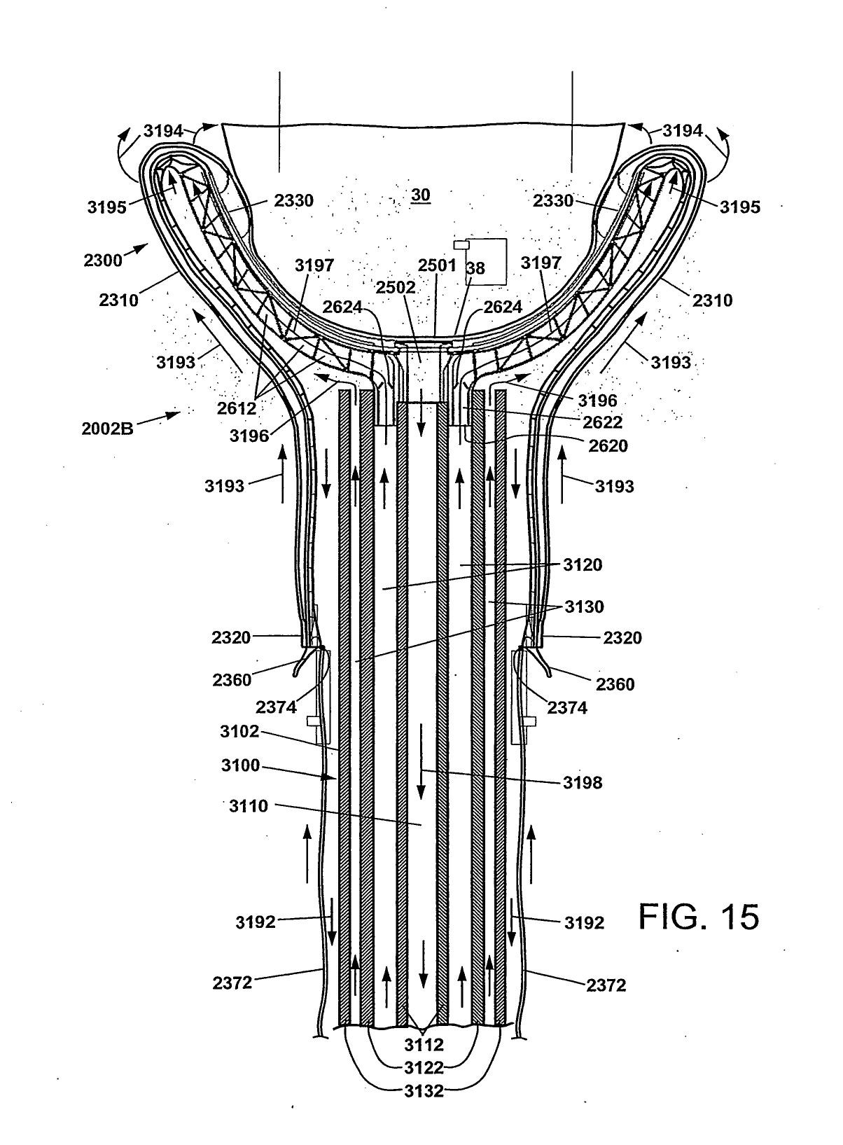

DMVA device 2002 is deployable from its collapsed state to a working state (depicted in FIG. 12) on a heart by a minimally invasive surgical procedure. Such deployment of DMVA device 2002 onto a heart is best understood with reference to FIGS. 14 and 15. FIG. 14 is a longitudinal cross-sectional view of the DMVA device of FIGS. 12 and 13 shown collapsed, inverted, and temporarily attached to an endoscopic inflation tool 3100 for deployment onto a heart; and FIG. 15 is a longitudinal cross-sectional view of the DMVA device 2002 and endoscopic inflation tool of FIG. 14 shown with the collapsed DMVA device 2002 partially deployed onto a heart.

Referring first to FIG. 14, collapsed DMVA device 2002A is temporarily attached to deployment and inflation tool 3100 at annular fitting 2620. Deployment and inflation tool 3100 is comprised of a multi-walled structure having a central tubular passageway 3110 aligned with central axis 3199, and one or more annular passageways around central tubular passageway 3110. In deployment and inflation of the DMVA device 2002A, these passageways are used cooperatively to inflate DMVA device 2002A, and deploy DMVA device 2002A over a heart 30, as will be explained below.

Referring again to FIG. 14, collapsed DMVA device 2002A is temporarily secured to tool 3100, such that port 2502 of fitting 2501 is aligned and in communication with central tubular passageway 3110 within tube 3112, and annular port 2622 of annular fitting 2620 is aligned and in communication with inner annular passageway 3120, which is formed between tube 3112 and tube 3122. In one embodiment, the dimensions of annular fitting 2620 and annular passageway 3120 are provided such that tool 3100 is secured to device 2002A by a mild interference fit between such parts, and tool 3100 may be separated from deployed DMVA device 2002 (see FIG. 12) by a twisting action.

The collapsed shell 2600 and liner 2300 are then inverted (i.e. turned “inside out”) and disposed over the length of tool 3100 as depicted in FIG. 14. The DMVA device 2002A is thus ready for deployment onto a patient's heart. Referring again to FIG. 14, and in one minimally invasive deployment procedure, access to a patient's heart 30 is obtained through incisions in the thorax and pericardium, and the distal end 3102 of tube 3132 is then placed through such incisions at the apex of the heart as described previously herein. For the sake of simplicity of illustration in FIGS. 14 and 15, shell 2600 and liner 2300 are depicted as being loosely draped over the length of tool 3100, with it being understood that when DMVA device 2002A on tool 3100 is inserted into an incision in the thorax of a patient and positioned proximate to the heart 30, the shell 2600 and liner 2300 of DMVA device 2002A will be pressed against the outer wall of tube 3132 of tool 3100, and that shell 2600 and liner 2300 will be in a folded or pleated state around tool 3100.

DMVA device 2002A is positioned proximate to the apex 38 of heart 30 as vacuum is applied to tubular passageway 3110 as indicated by arrow 3198, and thus to port 2502 of fitting 2501. Heart 30 is thus drawn toward DMVA device 2002A as indicated by arrow 97, and apex 38 of heart 30 then contacts fitting 2501 and is held in position by such vacuum as indicated in FIG. 15. In one embodiment, the upper surface of fitting 2501 may be provided with an adhesive such as, e.g., a cyanoacrylate adhesive to better secure the DMVA device 2002A to the heart during the deployment procedure.

Referring also now to FIG. 15, which depicts partially deployed DMVA device 2002B, a pressurized fluid is then delivered through inner annulus 3120 of tool 3100 through annular port 2622 of annular fitting 2620 as indicated by arrows 3197, inflating the interstitial spaces 2612 of shell 2600. In one embodiment, tool 3100 is provided with an outer annular passageway 3130 formed between tube 3122 and tube 3132. Pressurized sterile air or other suitable gas is delivered through annulus 3130 as indicated by arrows 3196, and this pressured gas provides a normal force on the outer wall of shell 2600, in particular at the area of inflation, as indicated by arrows 3195. This normal force, together with the inflation of shell wall 2610, results in a rolling of shell wall 2610 against heart 30 as indicated by arrows 3194, and an advancement of the shell wall 2610 upwardly along the heart 30 and along the outer wall 3102 of tool 3100, as indicated by arrows 3193.

The pressurized air provides some degree of inflation between shell wall 2610 and the outer surface of tube 3132 of tool 3100, to enable the shell wall 2610 to more easily slide along the outer surface of tube 3132 of tool 3100 during deployment of DMVA device 2002A/2002B. The pressurized air is vented out between shell wall 2610 and the outer surface tube 3132 of tool 3100 as indicated by arrows 3192. In order to prevent this vented air from escaping into the thorax of the patient, DMVA device 2002A/2002B is provided with a containment sleeve 2372, which is joined to the shell 2600 proximate to seal 2360. Containment sleeve 2372 extends out along tool 3100 to a location outside of the patient's body, so that the pressurized assisting gas is vented outside of the patient's body during the entire procedure. Containment sleeve 2372 also advances upwardly as DMVA device 2002A/2002B is deployed, until such device is fully deployed as indicated by DMVA device 2002 of FIG. 12, and even at the end of deployment, the outer end (not shown) of containment sleeve 2372 remains outside of the patient's body venting the assist gas.

Referring again to FIGS. 12, 14, and 15, and in one embodiment, there is provided a small resistive wire 2374 around the perimeter of containment sleeve 2372 in close proximity to the region at which containment sleeve 2372 is joined to shell 2600. When deployment of DMVA device 2002 is completed as indicated in FIG. 12, a short burst of electrical power is applied to wire 2374 through a pair of conductive leads (not shown). Wire 2374 heats and melts containment sleeve 2372 locally at wire 2374, allowing containment sleeve 2372 to be separated from DMVA device 2002 and withdrawn through the incision in the patient. In the embodiment depicted in FIG. 12, wire 2374 is shown as remaining attached to the device, with it being understood that alternatively, the melting may be made to occur such that wire 2374 remains attached to containment sleeve 2372, and is withdrawn from the patient.

After completion of the deployment of DMVA device 2002 onto heart 30, tool 3100 is detached from DMVA device 2002 and withdrawn from the patient. Annular fitting 2620 may be further provided with a check valve mechanism 2624, which prevents the backflow of any inflation fluid from DMVA device 2002A during deployment, and maintains shell 2600 in an inflated state when tool 3100 is removed after deployment, and during ongoing operation of DMVA device 2002. In further embodiments, check valve 2624 is either formed integrally as part of fitting 2600, or is fitted within annular passageway 2622. Referring to FIGS. 14, and 15, it can be seen that during deployment, check valve 2624 is open, allowing the passage of inflation fluid therethrough as indicated by arrows 3197, and after inflation and deployment of DMVA device 2002 as shown in FIG. 12, check valve 2624 is closed, thereby retaining the inflation fluid within the interstitial spaces 2612 of shell 2600.

In order to begin operation, DMVA device 2002 is then connected to operating vacuum and DMVA fluid drive lines. In one embodiment (not shown) DMVA device 2002 is provided with a multi-port fitting for the application of vacuum at the apex of the heart, and for the delivery and withdrawal of DMVA drive fluid. This multi-port fitting may be similar to the fitting 2551 of device 2000 of FIG. 9 previously described herein.

Referring again to FIG. 12, when shell wall 2610 is fully inflated, the matrix of pressurized interstitial spaces 2612 and walls 2614 therebetween provide the requisite structural strength required for the operation of DMVA device 2002. In one embodiment, shell wall 2610 is inflated with a gas such as air. In another embodiment (not shown), shell wall 2610 may be provided with a small venting tube at the top edge thereof near seal 2360, such that any gas within collapsed wall 2610 (see FIGS. 14 and 15) may be vented as such liquid advances up through shell wall 2610. Such liquid may be polymerizable such that when shell wall 2610 is fully inflated and filled with liquid and deployed on the patient's heart, such liquid cures to a solid state, thereby providing a shell wall with the desired properties. In another embodiment, such shell wall may be inflated with a liquid polymer foam that cures to a solid state. In another embodiment, shell wall 2610 may be filled with a hydrogel. Suitable hydrogels and the use thereof are disclosed in PCT International Publication Number WO 99/44665, “Medical device utilizing hydrogel materials,” the disclosure of which is incorporated herein by reference.

In another embodiment (not shown) of the deployment of DMVA device 2002 onto the heart, the shell and liner assembly may be rolled up into a toroidal shape, either being rolled inwardly, or outwardly. In such a configuration, the deployment tool 3100 of FIG. 14 would not require the provision of outer annulus 3130, and a higher inflation pressure would likely be required to drive the unrolling and inflation of the shell 2600 and attached liner 2300.

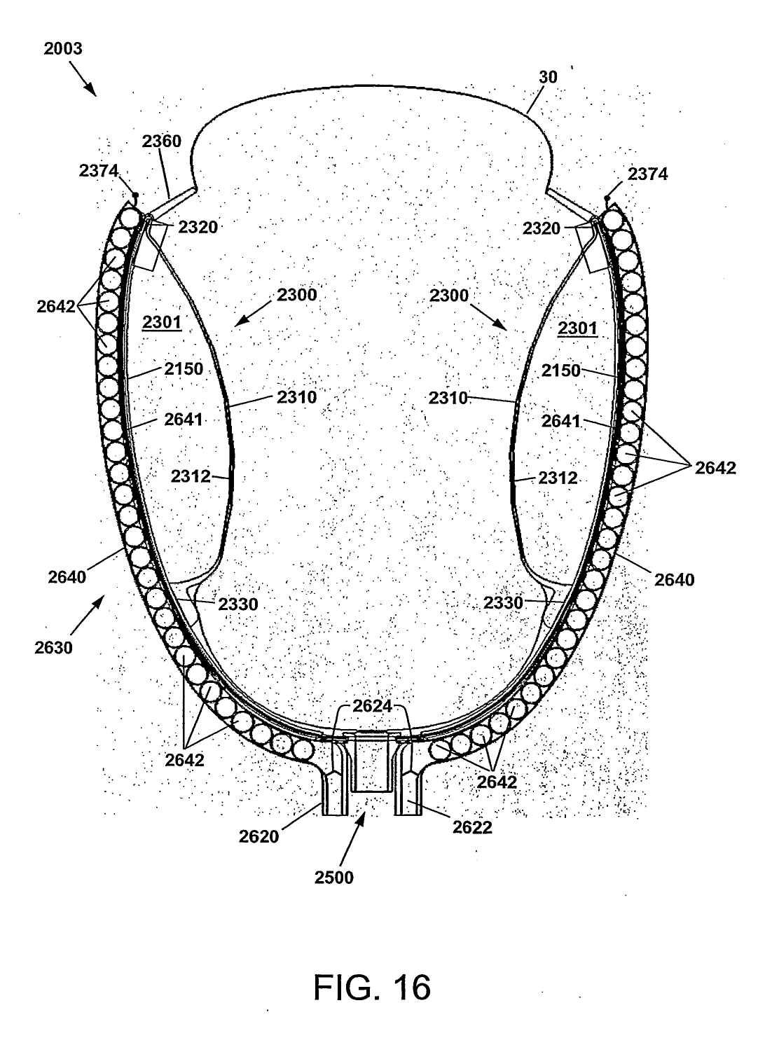

In an alternate embodiment, shell wall 2610 of the device of FIG. 12 may be comprised of a plurality of inflatable adjacent rings, instead of interstitial spaces formed within such wall. FIG. 16 is a side cross-sectional view of a further embodiment of a DMVA device 2003 similar to the DMVA device 2002 of FIG. 12, shown in a state of completing assistance of systolic compression of a heart. Referring to FIG. 16, DMVA device 2003 is comprised of structural shell 2630, which is used in lieu of shell 2600 of DMVA device 2002 of FIG. 12.

DMVA device 2003 comprises a cup-shaped shell 2630, a liner 2300 forming an inflatable cavity 2301 with the inner surface 2641 of wall 2640 of shell 2630 for actuation of the ventricles of a heart 30. Wall 2640 further comprises a plurality of inflatable rings 2642, which are inflated as previously described for the inflatable shell wall 2610 of DMVA device 2002 of FIG. 12. The remaining elements of DMVA device 2003 are substantially the same as for device 2002 of FIG. 12, and are numbered in FIG. 16 in the same manner as in FIG. 12. Hence these elements will not be described in further detail. DMVA device 2003 may be collapsed in the same manner as described for DMVA device 2002, and DMVA device 2003 may be inflated and deployed on a heart 30 in the same manner as described herein for DMVA device 2002 using deployment and inflation tool 3100 of FIGS. 14 and 15.

DMVA device 2003 may further comprise a small resistive wire 2374 for detaching a containment sleeve that is used during deployment of DMVA device 2003, as was previously described for DMVA device 2002 with reference to FIGS. 12, 14, and 15. In a further embodiment (not shown), one or more of the materials used in forming shell 2630 may contain radiopaque material that shields the heart from radiation during certain medical imaging procedures.

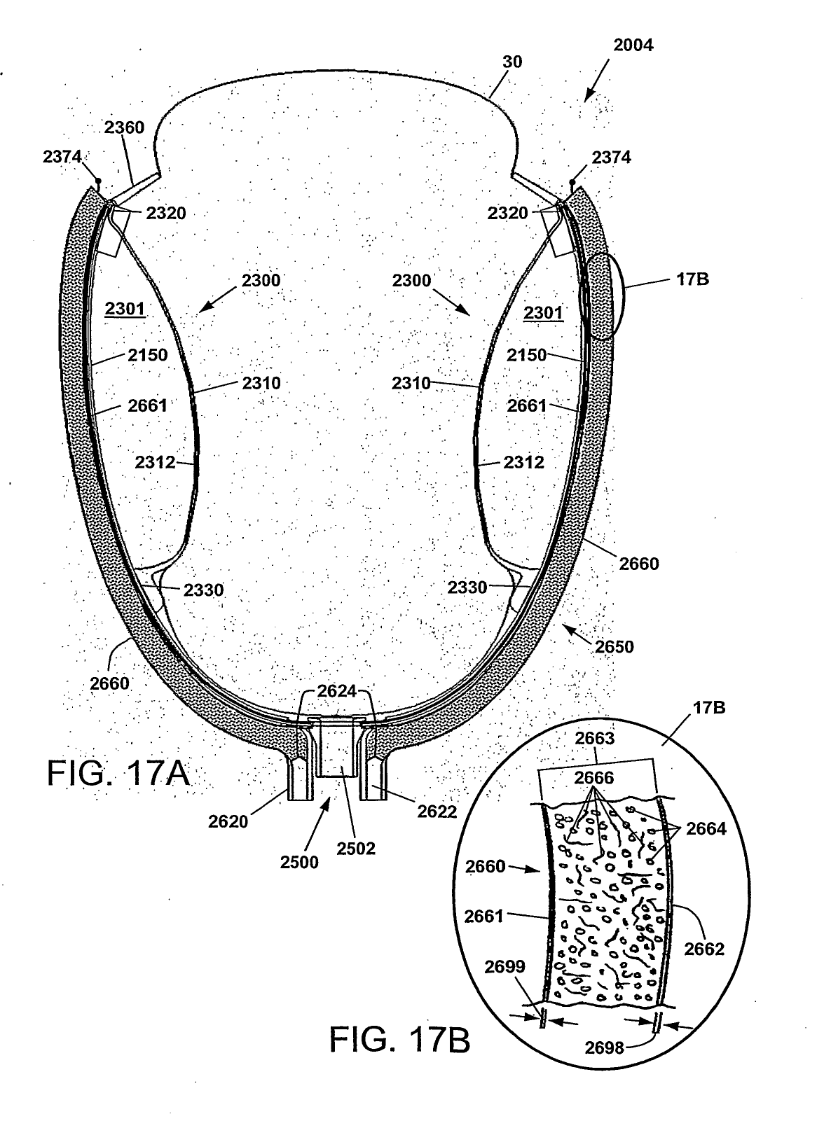

In another embodiment, shell wall 2610 of the device of FIG. 12 may be comprised of a reinforced foam composite wall, instead of interstitial spaces formed within a wall. FIG. 17A is a side cross-sectional view of another embodiment of a DMVA device 2004 similar to the DMVA device 2002 of FIG. 12, shown in a state of completing assistance of systolic compression of a heart. FIG. 17B is a detailed cross-sectional view of the shell wall of the device of FIG. 17A, a portion of which is denoted by the ellipse 17B of FIG. 17A. Referring to FIG. 17A, DMVA device 2004 is comprised of structural shell 2650, which is used in lieu of shell 2600 of DMVA device 2002 of FIG. 12.

DMVA device 2004 comprises a cup-shaped shell 2650, a liner 2300 forming an inflatable cavity with the inner surface 2661 of wall 2660 of shell 2650 for actuation of the ventricles of a heart 30. The remaining elements of DMVA device 2003 are substantially the same as for DMVA device 2002 of FIG. 12, and are numbered in FIG. 17A in the same manner as in FIG. 12. Hence these elements will not be described in further detail. DMVA device 2004 may be collapsed in the same manner as described for DMVA device 2002, and DMVA device 2004 may be inflated and deployed on a heart 30 in the same manner as described herein for DMVA device 2004 using deployment and inflation tool 3100 of FIGS. 14 and 15.

Wall 2660 further comprises an outer skin or layer 2662 and an inner skin or layer 2661, between which is formed a composite foam region 2663 comprised of a matrix of open cells 2664 and finely divided or chopped fibers 2666. Fibers 2666 are substantially fully wetted and embedded within the polymer resin forming the foam open cell matrix. Wall 2660 of DMVA device 2004 may be inflated as previously described for the inflatable shell wall 2610 of DMVA device 2002 of FIG. 12.

Wall 2660 has a relaxed or neutral shape that is free of stress, which is typically the shape assumed when the pressure within the cells 2664 is at atmospheric pressure. (Although wall 2660 could be made such that its stress-free condition is at a pressure greater than or less than atmospheric pressure.) When wall 2660 is inflated, and the pressure within the cells 2664 of wall 2660 exceed atmospheric pressure (or the pressure at which wall 2660 assumes its neutral shape), the walls and other interconnecting “bridge” regions between cells 2664 are caused to stretch. However, the embedded chopped fibers 2666 that are part of the foam composite region 2663 are inelastic fibers, and thus resist expansion of the cells 2664 and any overall expansion of the foam composite region 2663. Hence wall 2660 is substantially inelastic when foam composite region 2663 is inflated beyond a pressure at which fibers 2666 are brought into tension.

In one embodiment, shell walls 2661 and 2662 are formed with the silastic liquid silicone rubber previously described in this specification. Shell wall inner and outer layers 2661 and 2662 can have thicknesses 2699 and 2698 of between about 0.01 and 0.05 inches. In one embodiment, shell wall layers 2661 and 2662 have thicknesses 2699 and 2698 of about 0.020 inches. Foam composite region or core 2663 may be between about 0.25 inches and about 0.60 inches. In one embodiment, foam core 2663 is about 0.460 inches thick. In one embodiment, foam core 2663 may be formed of open cell urethane foam having a solid volume of about 4.5 percent, and containing 0.5 weight percent chopped strand KEVLAR™ fiber, a high strength, high rigidity para-aramid fiber manufactured and sold by the Dupont Company of Wilmington, Del.

DMVA device 2004 may be manufactured by a process which includes the steps of molding foam core 2663, fitting and/or bonding fitting 2500 to foam core 2663, coating foam core 2663 with inner and outer layers 2661 and 2662, bonding liner 2300 (optionally including seal 2360) to inner layer 2661 of shell 2650; and bonding seal 2360 to shell wall 2660 if seal 2360 is not integrally formed as part of liner 2300. It will be apparent that the order of steps may be varied somewhat; for example, inner and outer layers 2661 and 2662 may be applied to foam core 2663 prior to the addition of fitting 2500 to the DMVA device 2004.

Following the assembly of DMVA device 2004, such DMVA device 2004 may be evacuated to achieve full collapse of DMVA device 2004 to a minimum volume, and then placed in a deployment tool such as tool 3000 of FIG. 10A. The DMVA device 2004 together with the deployment tool 3000, or other suitable deployment tool (not shown) may be sterilized and packaged for subsequent use in an installation procedure.

For a DMVA device 2004 having an overall 5 inch inflated diameter of shell 2650, the 0.020 inch thick silastic inner and outer layers, the above recited 0.460 inch thick urethane/Kevlar™ foam core, and an elastic liner 2300 about 0.025 inches thick, such device may be evacuated and collapsed down to a cross-sectional area of about 1.5 square inches. Such DMVA device 2004 may be placed within a deployment tool, such as deployment tool 3000 of FIGS. 10A and 10B having an inside diameter of tube 3210 (See FIG. 10A) of about 1.37 inches, or an elliptical deployment tool of about 1.9 inches major axis by 1.0 inch minor axis.

Alternatively, DMVA device 2004 may be evacuated and deployed using inflation and deployment tool 3100 as shown for DMVA device 2002 in FIG. 14 and FIG. 15.

DMVA device 2004 may further comprise a small resistive wire 2374 for detaching a containment sleeve that is used during deployment of DMVA device 2004, as was previously described for DMVA device 2002 with reference to FIGS. 12, 14, and 15. In a further embodiment (not shown), one or more of the materials used in forming shell 2650 may contain radiopaque material that shields the heart from radiation during certain medical imaging procedures.

Deployment of DMVA device 2004 may be done by surgical procedures including a sub-xyphoid incision and approach, a subcostal incision and approach, or a mini-thoracotomy, optionally including ultrasound or video-assisted thorascopic imaging and guidance. In one procedure (not shown), a subcostal incision is made in the thorax, the unpackaged assembled device and deployment tool, such as deployment tool 3000 of FIG. 10A, is inserted into the incision and positioned near the apex of the heart. An ultrasonic probe inserted into the vacuum port 2502 (see FIG. 8A) or 2552 (see FIG. 9) may be used optionally to produce an image for guidance during the deployment procedure.

The shell core 2663 is pressurized though annular port 2662, while DMVA device 2004 is deployed outwardly from deployment tool 3000. Concurrently, vacuum is applied though vacuum port 2502 to assist in drawing device 2004 around the heart. When DMVA device 2004 is fully deployed upon heart 30 as depicted in FIG. 17A, operation of DMVA device 2004 to assist heart 30 is begun.

The foregoing steps may be performed in reverse order (without using deployment tool 3000) after making an incision in order to remove DMVA device 2004. In such a procedure, a fine wire may be inserted inwardly through the lumen connected to annular port 2622 sufficiently so as to break the seal provided by check valve 2624, enabling the evacuation and collapse of foam core 2663 of shell 2650.

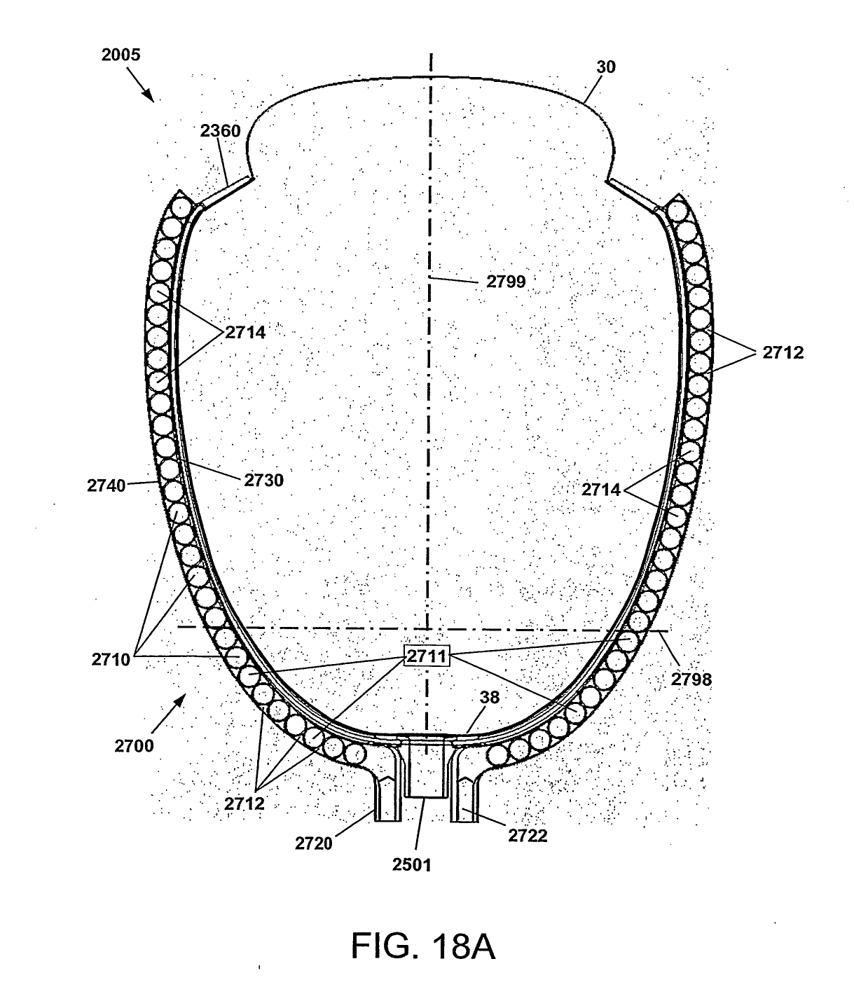

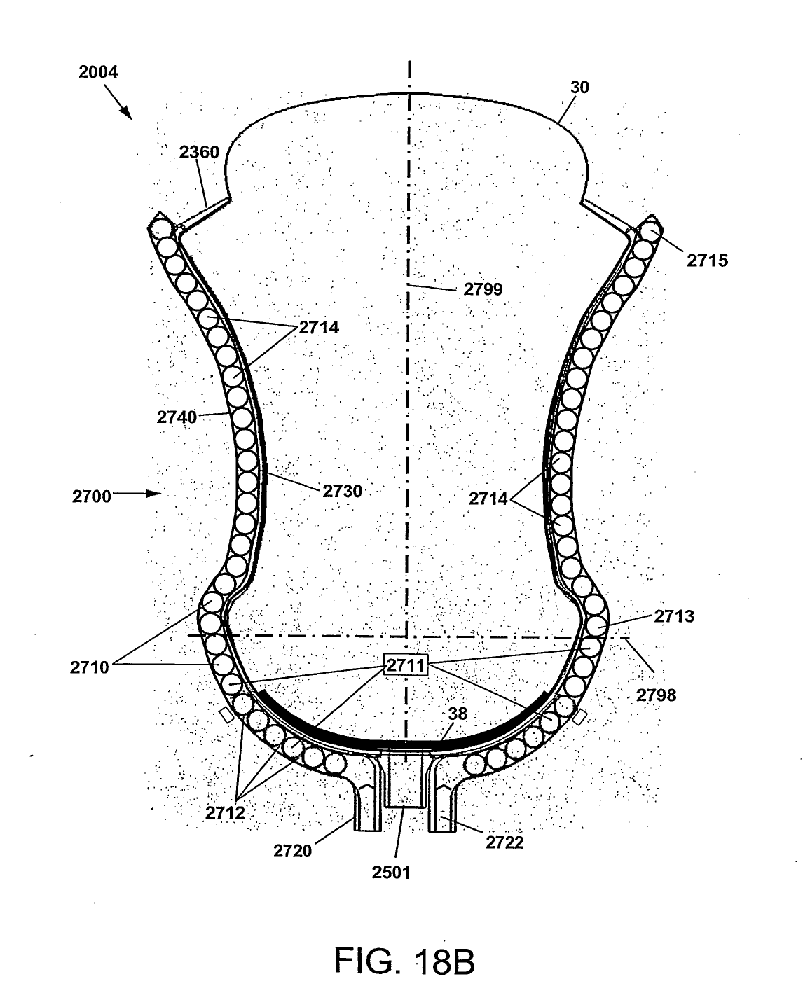

FIG. 18A is a side cross-sectional view of a further embodiment of a DMVA device of the present invention that is capable of being deployed onto a heart by a minimally invasive surgical procedure, shown in a state of completing assistance of diastolic expansion of a heart; and FIG. 18B is a side cross-sectional view of the of a DMVA device 2005 of FIG. 18A shown in a state of completing assistance of systolic compression of a heart. Referring to FIGS. 18A and 18B, DMVA device 2005 is comprised of a shell 2700 that includes a series of stacked coaxial rings 2710 centered around central axis 2799 disposed between an elastic inner wall 2730 and an outer elastic wall 2740. DMVA device 2005 further comprises a fitting connection 2501 for application of vacuum to the apex 38 of heart 30.

Coaxial rings 2711 located below the plane indicated by line 2798 are inactive support rings which provide support for the apical region of heart 30 when DMVA device 2005 is deployed on heart 30. Coaxial rings 2714 are active rings consisting essentially of an electrostrictive or electroactive polymer artificial muscle material (EPAM) such as e.g., a silicone EPAM or a polyurethane EPAM previously described in the specifications of the aforementioned U.S. patent application Ser. Nos. 10/607,434, and 10/795,098. Each of active rings 2714 are individually addressable, and the extent of constriction of each is controllable such that systolic assistance to the heart may be applied by constriction of rings 2713 through 2715 as indicated in FIG. 18B. DMVA device 2005 is deployable in a manner similar to that described for DMVA device 2002 of FIGS. 12-15 via inflation of the interstitial spaces 2712 between rings 2711 and 2714 through annular passageway 2722 in fitting 2720.

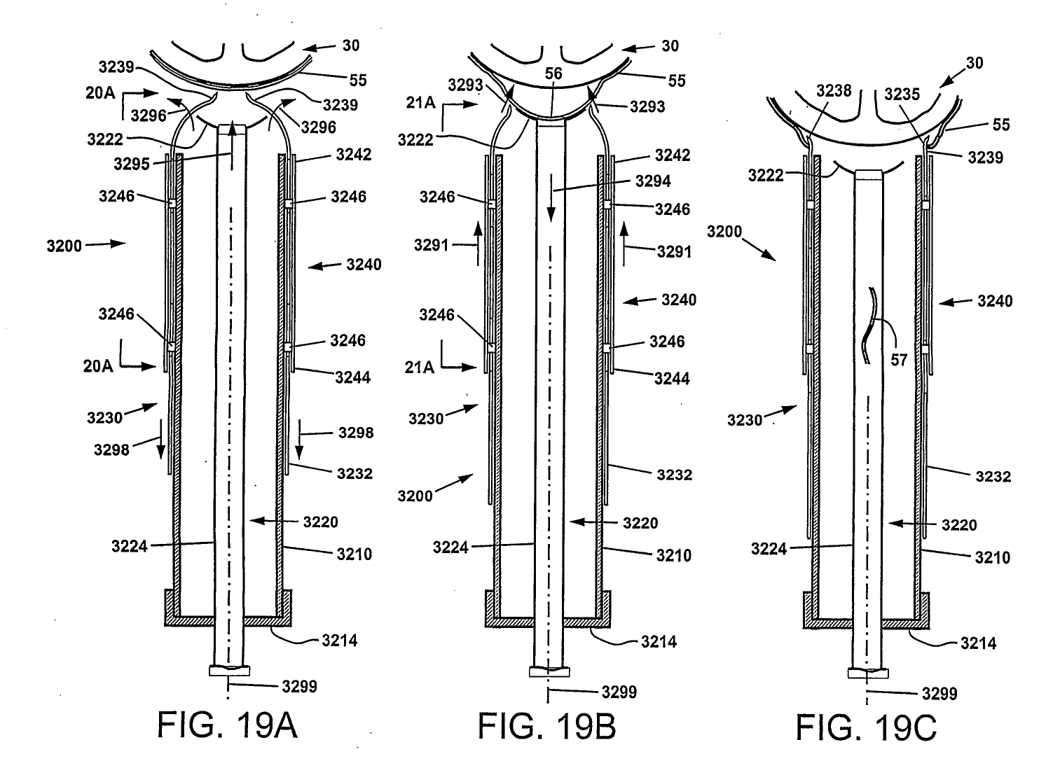

To obtain access to the heart for deployment of the minimally invasive DMVA devices described herein, there is provided an access tool for acquiring, cutting through, and opening the pericardium at the apex of the heart. The structure and use of an embodiment of such an access tool is best understood with reference to FIGS. 19A-22, which are summarized as follows:

FIG. 19A is a cross-sectional view of one embodiment of an access tool for gaining access to the apex of the heart throughout the pericardium, shown immediately prior to use. FIG. 19B is a cross-sectional view of the tool of FIG. 19A, shown pulling a portion of the pericardium from the heart, and cutting through the pericardium. FIG. 19C is a cross-sectional view of the tool of FIG. 19A, shown as having cut through and stretched the pericardium open, in order to provide access to the heart for deployment of a minimally invasive DMVA device as described previously, or for some other beneficial purpose.

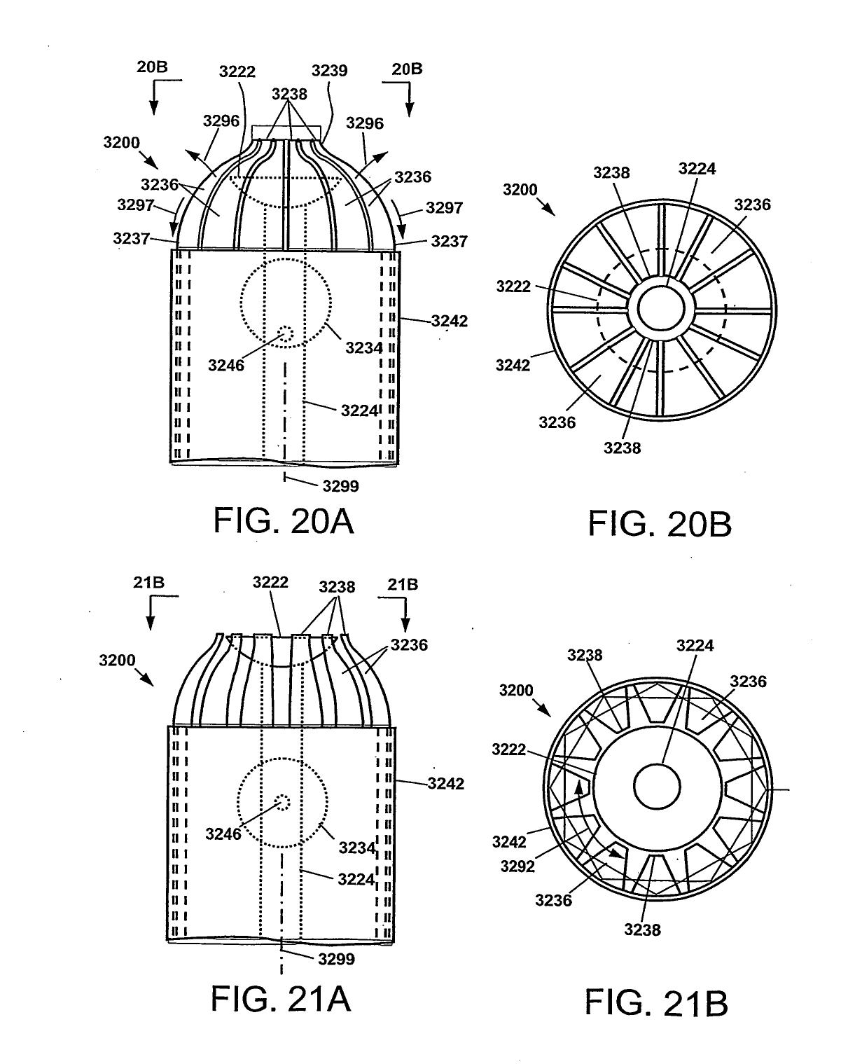

FIG. 20A is a detailed side elevation view of the portion of the access tool indicated by line 20A-20A in FIG. 19A, shown immediately prior to use. FIG. 20B is an end view of the access tool taken along line 20B-20B of FIG. 20A. FIG. 21A is a detailed side elevation view of the portion of the access tool indicated by line 21A-21A in FIG. 19B, shown deployed for pulling a portion of the pericardium from the heart, and cutting through the pericardium. FIG. 21B is an end view of the access tool taken along line 21B-21B of FIG. 20A.

Reference is first made to FIGS. 19A and 20A, wherein access tool 3200 is depicted as having been inserted through an incision in the thorax (not shown) as previously described, and positioned in close proximity to the heart 30. Access tool 3200 is comprised of tubular housing 3210, retainer cap 3214, suction tube assembly 3220, cutting sleeve 3230, and retainer sleeve 3240. Cutting sleeve 3230 comprises an elongated thin tubular section 3232 disposed in an annular gap formed between retainer sleeve 3240 and tubular housing 3210. Retainer sleeve 3240 is joined to tubular housing 3210 at upper end 3242 and lower end 3244 by several spot welds 3246 spaced around the circumference thereof. Cutting sleeve 3230 further comprises several corresponding cutouts 3234 positioned such that each of spot welds 3246 is enclosed within a corresponding cutout 3234. In this manner, cutting sleeve 3230 is provided with a limited range of motion both along the central axis 3299 of access tool 3200 and rotationally around tubular housing 3210 of access tool 3200. Although cutouts 3234 are depicted in FIGS. 20A, and 20B as being circular, it will be apparent that other shapes such as elliptical or rectangular would provide a suitable range of axial and rotational motion for cutting sleeve 3230.

Cutting sleeve further comprises a plurality of cutting blades 3236, each of which includes a cutting edge 3238 at the distal ends thereof. When access tool 3200 is placed in close proximity to heart 30 as shown in FIG. 19A, the surgeon moves cutting sleeve 3230 in the axial direction as indicated by arrows 3298. Cutting sleeve 3230 is formed of a material that is suitable for making surgical incisions, and cutting sleeve 3230 is made sufficiently thin in wall thickness so as to provide flexibility in cutting blades 3236. Cutting sleeve 3230 may be made of surgical stainless steel, nitinol, or another suitable metal alloy. Accordingly, when cutting sleeve 3230 is moved as indicated by arrows 3298, the proximal ends 3237 of cutting blades 3236 flex and are drawn into the annular gap between retainer sleeve 3240 and tubular housing 3210 as indicated by arrows 3297. The distal ends 3239 flex outwardly as indicated by arcuate arrows 3296. When this first step of accessing the heart 30 through the pericardium 55 is complete, the cutting edges 3238 of access tool 3200 are positioned as indicated in FIGS. 19B, 21A, and 21B, with the cutting edges 3238 forming a circle slightly larger in diameter than the diameter of suction cup 3222.

Subsequently, the surgeon advances suction tube assembly 3220 toward heart 30 as indicated by arrow 3295 in FIG. 19A. Suction cup 3222 is extended beyond cutting edges 3238 and brought into contact with the pericardium 55. A vacuum is applied from a vacuum source (not shown) that is connected to hollow suction tube 3224, thereby temporarily attaching suction cup 3222 to pericardium 55. Suction tube assembly 3220 is then withdrawn a short distance as indicated by arrow 3294 of FIG. 19B, pulling the apical region 56 of the pericardium 55 away from the apex of heart 30.

Cutting sleeve 3230 is now advanced by the surgeon as indicated by arrows 3293 so that cutting edges 3238 are brought into contact with the apical region 56 of the pericardium 55. The surgeon then rotates cutting sleeve 3230 such that cutting edges 3238 move against the pericardium 55 as indicated by arcuate arrow 3292 of FIG. 21B. When edges 3238 cut through the pericardium 55, the surgeon advances the tool 3200 toward the heart as indicated by arrows 3291 of FIG. 19B, while holding cutting sleeve 3230 close to heart 30. This motion results in the further spreading of the resulting cut hole in the pericardium 55 when cutting edges 3238 are splayed fully open as indicated in FIG. 19C. It can be seen that the outward curvature 3235 at the distal end 3239 of cutting blades 3236 assists in acquiring and spreading the pericardium 55 at the hole cut therein.

The cut circle 57 of pericardial tissue is captured within suction tube 3224. The heart has now been accessed by tool 3200 through the pericardium 55, and is ready for deployment of a minimally invasive DMVA device.

Access tool may be converted to a minimally invasive deployment tool that is similar to the deployment tool 3000 depicted in FIGS. 10A and 10B and previously described herein. Referring again to FIG. 19C, access tool 3200 is provided with retainer cap 3214, which is removable from tubular housing 3210. Retainer cap 3214 may be provided with threads (not shown) for engaging and securing to corresponding thread (not shown) on tubular body 3210. Alternatively, retainer cap 3214 and tubular body 3210 may be formed with other quick-disconnect fastening means, such as the bayonet lug-and-channel configuration of a Bayonet Neill Concelman (BNC) connector.

To perform a rapid conversion of access tool 3200 to a minimally invasive deployment tool, retainer cap 3214 is removed from tubular body 3210 by the surgeon or other practitioner, and the suction tube assembly 3220 is also removed. In place of these components, a second assembly is inserted into tubular body that includes a second retainer cap, a deployment sleeve, a piston and plunger rod, and the DMVA device disposed in the deployment sleeve.

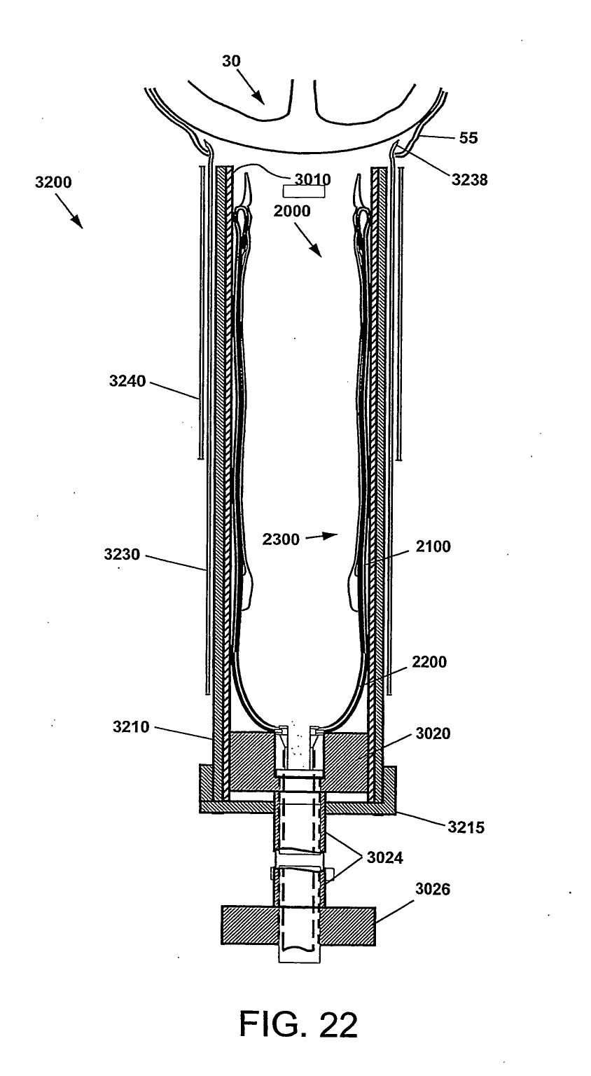

FIG. 22 is a side cross-sectional view of the access tool 3200 of FIGS. 19A-21B, with the pericardial suction tube assembly 3220 having been removed, and replaced by a sleeve and plunger assembly containing a minimally invasive DMVA device. Referring to FIG. 22, the subcomponents of deployment tool 3000, including deployment sleeve 3010, piston 3020, and plunger rod 3024 have been fitted within tubular housing 3210, and retained by replacement retainer cap 3215. Minimally invasive DMVA device 2000, or another DMVA device described herein is contained within deployment sleeve 3010 and ready for deployment onto heart 30 as previously described herein.

It is, therefore, apparent that there has been described herein an apparatus for direct mechanical ventricular assistance to a heart, the apparatus being deployable onto the heart by a minimally invasive surgical procedure. While certain embodiments have been described in detail, it is evident that many alternatives, modifications, and variations will be apparent to those skilled in the art. Accordingly, it is intended to embrace all such alternatives, modifications and variations that fall within the spirit and broad scope of the appended claims.

We claim:

1. A device for assisting the function of a heart in a body comprising:
a. a cup-shaped shell having a wall with an outer surface and an inner surface, the wall extending from an apex of the shell to a rim;
b. a collapsible cage disposed within the cup-shaped shell, the cage having a plurality of struts extending along the inner surface of the shell; and
c. a liner comprising an outer surface, an inner surface, an upper region joined to the cup-shaped shell, an elastic central region, and a lower region joined to the cup-shaped shell forming a cavity between the liner and the shell.

2. The device of claim 1, further comprising:
a. a first fitting in fluid communication with the interior of the cup-shaped shell; and
b. a second fitting in fluid communication with the cavity between the liner and the shell.

3. The device of claim 1, wherein the first fitting at the apex of the cup-shaped shell is comprised of a tubular body, and a passageway extending from a distal end of the first fitting to the proximal end of the first fitting.

4. The device of claim 3, wherein the first fitting is formed integrally with the liner.

5. The device of claim 1, wherein the first fitting and the second fitting are formed as a single unitary fitting.

6. The device of claim 5, wherein the unitary fitting is comprised of a tubular body comprising a first passageway and a second passageway.

7. The device of claim 6, wherein the unitary fitting is formed integrally with the liner.

8. The device of claim 1, wherein the liner further comprises a tapered seal extending inwardly from the upper region toward the longitudinal axis of the cup-shaped shell.

9. The device of claim 1, wherein the cup-shaped support cage is joined to the cup-shaped shell by adhesive.

10. The device of claim 1, wherein the cup-shaped support cage is joined to the cup-shaped shell by a bonding layer on the inner surface of the cup-shaped shell.

11. The device of claim 1, wherein the number of the struts of the collapsible cage is between 8 and 32.

12. The device of claim 1, wherein the number of the radial struts of the cup-shaped support cage is 16.

13. The device of claim 1, wherein the distal ends of the radial struts extend to the rim of the cup-shaped shell.

14. The device of claim 13, wherein at least one of the radial struts of the cup-shaped support cage comprises an engagement feature at the distal end of at least one of the radial struts.

15. The device of claim 1, wherein the rim of the shell is comprised of an annular chamber.

16. The device of claim 1, wherein the device is collapsible along a longitudinal axis to a diameter of less than about 4 cm.

17. The device of claim 1, wherein the device is collapsible along a longitudinal axis to a diameter of less than about 2 cm.

18. The device of claim 1, wherein the struts of the collapsible cage are formed of an alloy selected from the group consisting of titanium and alloys thereof; tantalum and alloys thereof, stainless steel, carbon fiber composites, aramid fiber composites, and glass fiber composites.

19. The device of claim 1, wherein the polymer-fiber composite of the cup-shaped shell is a combination of polyester fiber and polyurethane polymer.

20. The device of claim 1, wherein the fiber of the polymer fiber composite is wound circumferentially around the shell to form a fiber matrix of substantially uniform fiber density.

21. The device of claim 1, wherein the fiber of the polymer fiber composite is chopped fiber forming a fiber matrix of substantially uniform fiber density.

22. The device of claim 1, wherein the fiber of the polymer fiber composite is formed of a woven mesh fabric.

23. The device of claim 1, wherein the liner is a silastic elastomer.

24. The device of claim 1, wherein the shell has a diameter of about 80 to about 140 millimeters and wherein the distance along the longitudinal axis from the apex to the rim of the shell is approximately equal to the diameter of the shell.

25. A device for assisting the function of a heart in a body comprising:
a. a cup-shaped shell having a wall with an outer surface and an inner surface, the wall extending from an apex of the shell to a rim;
b. a collapsible cage disposed within the cup-shaped shell, the cage having a plurality of struts extending along the inner surface of the shell;
c. a liner comprising an upper region joined to the rim of the cup-shaped shell, an elastic central region including an inflatable cavity, and a lower region joined to the cup-shaped shell;
d. a first fitting in fluid communication with the interior of the cup-shaped shell; and
e. a second fitting in communication with the inflatable cavity in the elastic central region of the liner.

26. A method of deploying a device of claim 1, on a heart in a body comprising:
a. collapsing the shell from an open cup-shape to a compact longitudinally collapsed shape;
b. inserting the collapsed shell into a deployment tool;
c. making an incision in the body proximate the heart;
d. inserting the tubular deployment tool through the incision;
e. displacing the collapsed shell from the deployment tool, and restoring the open cup-shaped configuration; and
f. positioning the shell over a heart disposed within a body.

27. The method of claim 26, wherein the deployment tool comprises a hollow tubular portion.

28. The method of claim 26, wherein the deployment tool comprises a cutting device on a leading edge of the tubular portion.
